(19) United States
(12) Patent Application Publication
(10) Pub. No.: US 2014/0147419 A1
(43) Pub. Date: May 29, 2014

(54) COMPOSITIONS AND METHODS FOR FORMATION OF BONE TISSUE

(71) Applicant: THE TRUSTEES OF COLUMBIA UNIVERSITY IN THE CITY OF NEW YORK, New York, NY (US)

(72) Inventors: Gordana V. Novakovic, New York, NY (US); Darja Marolt, Ljublijana (SI)

(73) Assignee: THE TRUSTEES OF COLUMBIA UNIVERSITY IN THE CITY OF NEW YORK, New York, NY (US)

(21) Appl. No.: 13/882,406

(22) PCT Filed: Oct. 28, 2011
(86) PCT No.: PCT/US11/58426

§ 371 Date: Dec. 20, 2013

Related U.S. Application Data
(60) Provisional application No. 61/408,534, filed on Oct. 29, 2010.

(51) Int. Cl. C12N 5/0775; A61K 35/32

(52) U.S. Cl. CPC C12N 5/0662; A61K 35/32
USPC 424/93.7; 435/377; 435/366

(57) ABSTRACT

Provided is a step-wise method of forming an engineered construct. Various embodiments include inducing differentiation of progenitor cells, such as embryonic stem cells; expanding the differentiated progenitor cells; combining the expanded progenitor cells and a biocompatible scaffold comprising a matrix material; and incubating the progenitor cells and the biocompatible scaffold so as to form a bone tissue module.

### CROSS-REFERENCE TO RELATED APPLICATIONS

The present application claims the benefit of U.S. Provisional Application Ser. No. 61/408,534 filed Oct. 29, 2010; which is incorporated herein by reference in its entirety.

### STATEMENT REGARDING FEDERALLY SPONSORED RESEARCH OR DEVELOPMENT

This invention was made with government support under R01 DE016525 awarded by NIH-NIDCR and P41EB002520 awarded by NIH-NIBIB. The government has certain rights in the invention.

### FIELD OF THE INVENTION

The present disclosure generally relates to engineered bone tissue.

### BACKGROUND OF THE INVENTION

Clinical needs of tissue grafting for the reconstruction of trauma, chronic diseases, tumor removal and congenital anomalies are substantial. Current surgical procedures rely on autologous grafts, allogenic grafts, xenogenic grafts or synthetic materials. The deficiencies associated with current clinical procedures are widely recognized in surgical and scientific communities.

Repair of large bone defects remains limited by the ability to harvest and shape autologous bone grafts, or by the non-biological nature of bone substitutes. Bone tissue engineering can provide an unlimited supply of functional viable bone grafts. Human embryonic stem (ESC) cells, and the embryonic-like iPS cells, represent a promising cell source for this goal, as they can: (i) grow indefinitely, providing unlimited numbers of tissue repair cells, and (ii) give rise to any cell type in the body. Osteogenic cells have previously been derived from ESC (see e.g., de Peppo G M, et al. Tissue Eng Part A, 2010, 16(11):3413), however their ability to form three-dimensional bone tissue in vitro or in vivo has not been demonstrated (see e.g., Kim S, et al. Biomaterials, 2008, 29(8):1043; Kuznetsov S A, et al. Stem Cells Dev, 2011, 20(2):269).

### SUMMARY OF THE INVENTION

Among the various aspects of the present disclosure is the provision of a method for forming a bone tissue module.

One aspect provides a method of forming a bone tissue module. In some embodiments, the method includes inducing differentiation of progenitor cells to form osteogenic progenitor cells; expanding the osteogenic progenitor cells; combining the osteogenic progenitor cells and a biocompatible scaffold comprising a matrix material; and incubating the osteogenic progenitor cells and the biocompatible scaffold so as to form a bone tissue module. In some embodiments, the method includes inducing differentiation of embryonic stem cells (ESCs) to form mesenchymal-like progenitor cells; expanding the mesenchymal-like progenitor cells; combining the expanded mesenchymal-like progenitor cells and a biocompatible scaffold comprising a matrix material; and incubating the expanded mesenchymal-like progenitor cells and the biocompatible scaffold so as to form a bone tissue module.

In some embodiments, the method includes incubating the osteogenic progenitor cells and the biocompatible scaffold in vitro in a bioreactor.

In some embodiments, the progenitor cells comprise cells selected from the group consisting of mesenchymal stem cells (MSC), MSC-derived cells, embryonic stem cells, bone marrow stromal/stem cells, osteoblasts, and induced pluripotent cell lines. In some embodiments, the osteogenic progenitor cells comprise cells selected from the group consisting of mesenchymal stem cells (MSC), MSC-derived cells, embryonic stem cells, bone marrow stromal/stem cells, osteoblasts, and induced pluripotent cell lines. In some embodiments, the progenitor cells comprise embryonic stem cells. In some embodiments, the progenitor cells comprise induced pluripotent stem cells. In some embodiments, the progenitor cells comprise human progenitor cells.

In some embodiments, the matrix comprises decellularized bone. In some embodiments, the matrix comprises a material selected from the group consisting of fibrin, fibrinogen, a collagen, a polyorthoester, a polyvinyl alcohol, a polyamide, a polycarbonate, a polyvinyl pyrrolidone, a marine adhesive protein, a cyanoacrylate, a polymeric hydrogel, and a combination thereof.

In some embodiments, the biocompatible scaffold comprises progenitor cells at a density of at least about 0.0001 million cells (M) ml⁻¹ up to about 1000 M ml⁻¹. In some embodiments, the biocompatible scaffold comprises progenitor cells at a density of about 1 M ml⁻¹, about 5 M ml⁻¹, about 10 M ml⁻¹, about 15 M ml⁻¹, about 20 M ml⁻¹, about 25 M ml⁻¹, about 30 M ml⁻¹, about 35 M ml⁻¹, about 40 M ml⁻¹, about 45 M ml⁻¹, about 50 M ml⁻¹, about 55 M ml⁻¹, about 60 M ml⁻¹, about 65 M ml⁻¹, about 70 M ml⁻¹, about 75 M ml⁻¹, about 80 M ml⁻¹, about 85 M ml⁻¹, about 90 M ml⁻¹, about 95 M ml⁻¹, or about 100 M ml⁻¹. In some embodiments, the biocompatible scaffold comprises progenitor cells at a density of at least about 30 M ml⁻¹.

In some embodiments, inducing differentiation of progenitor cells comprises incubating progenitor cells in a differentiation medium. In some embodiments, the differentiation medium comprises one or more of DMEM, serum, dexamethasone, b-glycerophosphate, ascorbic acid, bone morphogenic protein, vitamin D, and pen-strep.

In some embodiments, expanding the osteogenic progenitor cells comprises culturing the osteogenic progenitor cells in an expansion medium. In some embodiments, the expansion medium comprises one or more of DMEM, serum, KO-serum replacement, nonessential amino acids, glutamine, b-mercaptoethanol, and bFGF.

In some embodiments, incubating in a bioreactor occurs at a superficial flow velocity of between about 80 μm/s and about 2,000 μm/s. In some embodiments, incubating in a bioreactor occurs at a superficial flow velocity of about 80 μm/s, about 100 μm/s, about 200 μm/s, about 300 μm/s, about 400 μm/s, about 500 μm/s, about 600 μm/s, about 700 μm/s, about 800 μm/s, about 900 μm/s, about 1,000 μm/s, about 1,100 μm/s, about 1,200 μm/s, about 1,300 μm/s, about 1,400 μm/s, about 1,500 μm/s, about 1,600 μm/s, about 1,700 μm/s, about 1,800 μm/s, about 1,900 μm/s, or about 2,000 μm/s. or (ii) about 80 μm/s, about 100 μm/s, about 200 μm/s, about 300 μm/s, about 400 μm/s, about 500 μm/s, about 600 μm/s, about 700 μm/s, about 800 μm/s, about 900 μm/s, about 1,000 μm/s, about 1,100 μm/s, about 1,200 μm/s, about 1,300 μm/s, about 1,400 μm/s, about 1,500 μm/s, about 1,600 μm/s, about 1,700 μm/s, about 1,800 μm/s, about 1,900 μm/s, or about 2,000 μm/s. In some embodiments, incubating in a bioreactor occurs at a superficial flow velocity of about 400 μm/s to about 800 μm/s.

In some embodiments, incubating the osteogenic progenitor cells and the biocompatible scaffold comprises incubating the osteogenic progenitor cells and the biocompatible scaffold in an osteogenic medium. In some embodiments, the osteogenic medium comprises DMEM, FBS, beta-glycerophosphate, dexamethasone and ascorbate-2 phosphate. In some embodiments, the osteogenic medium comprises DMEM, dexamethasone, ascorbate-2 phosphate, proline, ITS supplement, sodium pyruvate, TGFB3, and pen-strep.

Another aspect provides a method of treating a bone tissue defect. In some embodiments, the method includes grafting a bone tissue module produced according to a method described above into a subject in need thereof. In some embodiments, the subject is a mammalian subject. In some embodiments, the subject is a horse, cow, dog, cat, sheep, pig, rabbit, goat, chicken, or human. In some embodiments, the bone tissue defect comprises at least one of arthritis; osteoarthritis; osteoporosis; osteochondrosis; osteochondritis; osteogenesis imperfecta; osteomyelitis; osteophytes; achondroplasia; costochondritis; chondroma; chondrosarcoma; herniated disk; Klippel-Feil syndrome; osteitis deformans; osteitis fibrosa cystica, a congenital defect that results in absence of a tissue; accidental tissue defect or damage; fracture; wound; joint trauma; an autoimmune disorder; diabetes; Charcot foot; cancer; tissue resection; periodontal disease; implant extraction; or tumor resection. In some embodiments, the bone tissue module does not induce any substantially abnormal growth in the subject.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is an illustration of the bone engineering protocol and timeline. Undifferentiated hESC were cultured in mesoderm-inducing medium for 1 week. Adherent cells were expanded in monolayer for 4 passages (3-4 weeks) and seeded on bovine bone scaffolds in osteogenic medium for 3 days to allow cell attachment. Cell-seeded constructs were then cultured for 5 weeks in perfusion bioreactors or in static dishes in osteogenic medium. Tissue development was evaluated after 3 and 5 weeks of culture. Bioreactor-engineered bone was transplanted subcutaneously in SCID-beige mice for 8 weeks to evaluate tissue stability and maturation.

FIG. 2 is a line and scatter plot and a series of images showing morphology and proliferation growth characteristics of H9 hESCs for passages 2-11 (P2-P11). FIG. 2A shows cumulative cell number (millions) as a function of culture time (days). FIG. 2B is an image of hESCs at P4. FIG. 2C is an image of hESCs at P8. FIG. 2D is an image of hESCs at P10.

FIG. 3 is a line and scatter plot and a pair of images showing growth and morphology of ESC-derived progenitors. FIG. 3A is a line and scatter plot of cumulative cell number versus days of culture (up to 11 and 10 passages) in H9 progenitor and H13 progenitor. FIG. 3B is an image of the H9 progenitor at 100× magnification. FIG. 3C is an image of the H13 progenitor at 100× magnification.

FIG. 4 is series of graphs showing surface antigen expression for P1-P9. Immunophenotype of H9-derived progenitors is shown for P7. Expression pattern was similar to adult MSC.

FIG. 5 is a series of images and a bar graph showing adipogenesis and chondrogenesis. FIG. 5A-B show lipid droplets stained with Oil Red for H9 hESC progenitors. FIG. 5C-D show lipid droplets stained with Oil Red for BMSC. FIG. 5E-F show glycosaminoglycans (GAGs) stained with Alcian Blue in hESC progenitors and BMSC, respectively, under chondrogenic treatment. FIG. 5G-H show glycosaminoglycans (GAGs) stained with Alcian Blue hESC H9 progenitors and BMSC, respectively, under control conditions. FIG. 5I shows GAG/DNA (μg/μg) for hESC progenitors and BMSC with control, osteogenic treatment, and chondrogenic treatment.

FIG. 6 is a series of images showing osteogenesis in monolayer culture. FIG. 6A-D are H9 hESC progenitor cells. FIG. 6E-H are bone marrow stem cells (BMSC). FIGS. 6A, B, E, and F are tissues stained with alkaline phosphatase (blue). FIGS. 6C, D, G, and H are tissues stained with Von Kossa to stain calcified matrix (black).

FIG. 7 is a series of images and a bar graph showing osteogenesis in pellet culture. FIGS. 7A and B show H9 hESC progenitors with osteogenic treatment and control, respectively. FIGS. 7C and D show BMSC progenitors with osteogenic treatment and control, respectively. FIG. 7E shows calcium/DNA (μg/μg) for H9 hESC progenitors and BMSC with control, osteogenic treatment, and chondrogenic treatment.

FIG. 8A-C is a series of bar graphs and images showing mesenchymal differentiation potential of ESC-progenitors and BMSC. FIG. 8A shows osteogenic potential shown with alkaline phosphatase- and von Kossa-staining on sections fixed cultures and of BMSC1, BMSC2, H13-progenitors, and H9-progenitors. Inset figures show control conditions. Bar graphs show calcium content measured by biochemical analyses in control (Ctrl), osteogenic (Ost), and chondrogenic (Chond) media. FIG. 8B shows chondrogenic potential with a series of images of Alcian blue-stained pellet sections of BMSC1, BMSC2, H13-progenitors, and H9-progenitors. Inset figures show control conditions. Bar graphs show glycosaminoglycans content measured by biochemical analyses in control (Ctrl), osteogenic (Ost), and chondrogenic (Chond) media. FIG. 8C shows adipogenic potential with a series of images of oil red O-stained fixed cultures of BMSC1, BMSC2, H13-progenitors, and H9-progenitors. Inset figures show control conditions. Biochemical data represent averages of 3-6 measurements±standard deviation (p<0.05; * represents a statistically significant difference from other groups).

FIG. 9 is a pair of bar graphs, a line and scatter plot, and a series of images that show the effect of bioreactor cultivation on tissue development. FIG. 9A-C shows osteogenesis of ESC(H9) and BMSC. FIG. 9A is a bar graph of DNA content per wet weight (ww) of tissue constructs (expressed as percent initial value at the start of bioreactor/static cultivation) with respect to bioreactor groups (br) compared to the static group (st). FIG. 9B is a bar graph of alkaline phosphatase (AP) activity of H9 (static and bioreactor) and BMSC (bioreactor) at week 3 and week 5. FIG. 9 C is a line and scatter plot of cumulative osteopontin (OPN) release with respect to medium change. Data represent averages of 3-5 measurements±standard deviation (p<0.05; * and # represent statistically significant differences from the H9 bioreactor and BMSC bioreactor groups at the same time point; $ represents a statistically significant difference within the group between week 3 and week 5). FIG. 9D is a series of images of H&E and Masson Trichrome stained sections.

FIG. 10 is a series of images showing matrix formation (H&E). FIG. 10A-C show hESCP static, hESCP perfusion, and BMSC perfusion, respectively, at week 3. FIG. 10D-F show hESCP static, hESCP perfusion, and BMSC perfusion, respectively, at week 5. Scale bar represents 1 mm. Images are full sections of 4×4 mm tissues.

FIG. 11 is a series of images showing collagen deposition (Masson Trichrome staining). FIG. 11A-C show H9 hESCP static, H9 hESCP perfusion, and BMSC perfusion, respectively, at week 3. FIG. 11D-F show H9 hESCP static, H9 hESCP perfusion, and BMSC perfusion, respectively, at week 5. Scale bar represents 1 mm. Images are full sections of 4×4 mm tissues.

FIG. 12. is a pair of bar graphs, a line and scatter plot and a series of images that show the effect of bioreactor cultivation on tissue development from H13 progenitors. FIG. 12A is a bar graph of % initial DNA ww for H13 progenitors cultured in static or bioreactor conditions at week 3 and week 5. FIG. 12B is a bar graph of AP activity for H13 progenitors cultured in static or bioreactor conditions at week 3 and week 5. FIG. 12C is a line and scatter plot of the quantity of cumulative OPN release with respect to medium change in H13 progenitors cultured in static or bioreactor conditions. FIG. 12A-C data represent averages of 2-4 measurements±standard deviation (p<0.05; * represents a statistically significant difference between the groups; $ represents a statistically significant difference between week 3 and week 5). FIG. 12D shows a series images of histology slides of H13 progenitors in static cultivation and in bioreactor cultivation. Both environments were imaged at week 3 and week 5 stained with H&E at low-magnification (scale bar=500 μm) and Masson Trichrome at low-magnification (scale bar=500 μm) and high-magnification (scale bar=100 μm).

FIG. 13 is a series of images showing H9 progenitor cell survival in bone constructs. FIG. 13A shows day 1. FIG. 13B shows day 3. FIG. 13C shows week 3 static. FIG. 13D shows week 3 perfusion. FIG. 13E shows week 5 static. FIG. 13F shows week 5 perfusion.

FIG. 14 is a series of images of bioreactor-engineered bone tissue. H9 and BMSC were stained for positive for bone markers ostepontin (first row), bone sialoprotein (second row) and osteocalcin (third row). Insets represent negative staining controls. H9 and BMSC stained positive for osteoid deposition (Goldner's trichrome stain, fourth row).

FIG. 15 is a series of low-magnification images showing homogenous expression of bone markers (osteopontin, bone sialoprotein, and osteocalcin) in engineered tissue in H9 progenitors (in static and bioreactor cultivation conditions) and BMSCs (in a bioreactor culture condition) at 3 and 5 weeks.

FIG. 16 is a series of high-magnification images showing expression of bone markers in engineered tissue from H13 progenitors. Microscopy images of sections of H13 progenitors were stained for osteopontin, bone sialoprotein, and osteocalcin expression and stained with Goldner Trichrome for osteoid deposition. Insets represent negative staining controls.

FIG. 17 is a series of low-magnification images showing the expression of bone markers in engineered tissue from H13 progenitors. Microscopy images of sections of H13 progenitors were stained for osteopontin, bone sialoprotein, and osteocalcin expression in static and bioreactor culture conditions at 3 and 5 weeks. Insets represent negative staining controls.

FIG. 18 is a series of images showing osteopontin stain. FIG. 18A-C show H9 hESCP static, H9 hESCP perfusion, and BMSC perfusion, respectively, at week 3. FIG. 18D-F show H9 hESCP static, H9 hESCP perfusion, and BMSC perfusion, respectively, at week 5. Scale bar represents 1 mm. Images are full sections of 4×4 mm tissues.

FIG. 19 is a series of images showing bone sialoprotein stain. FIG. 19A-C show hESCP static, hESCP perfusion, and BMSC perfusion, respectively, at week 3. FIG. 19D-F show hESCP static, hESCP perfusion, and BMSC perfusion, respectively, at week 5. Scale bar represents 1 mm. Images are full sections of 4×4 mm tissues.

FIG. 20. is a series of reconstructed 3D μCT images and a series of bar graphs of engineered bone mineralization. FIG. 20A show reconstructed 3D μCT images of the tissue engineered bone constructs from H9-progenitors and BMSC before cultivation, after 5 weeks of cultivation, and after 8 weeks of in vivo transplantation indicating formation of mineralized tissue in all groups. FIG. 20B shows bar graphs of bone structural parameters (determined by μCT analysis) that indicate bone maturation during in vitro culture and in vivo implantation in H9-progenitors and BMSC. Bone structural parameters determined by μCT include: bone volume (BV), bone volume fraction (BV/TV), trabecular number (Tb.N.), trabecular thickness (Tb.Th.), trabecular spacing (Tb.Sp.), and connectivity density (Conn.D.). Data represent averages of 4 measurements±standard deviation (p<0.05; * and $ represent statistically significant differences from initial values and from week 5 values within the same group).

FIG. 21 is a series of reconstructed 3D μCT images and a series of bar graphs showing engineered bone mineralization from H13 progenitors. FIG. 21A shows reconstructed 3D μCT images of the tissue engineered bone constructs from H13 progenitors before and after 5 weeks of cultivation indicating formation of mineralized tissue. FIG. 21B shows bone structural parameters determined by μCT analysis and indicates bone maturation during in vitro culture.

FIG. 22 is a series of bar graphs and histology images showing the stability of engineered bone in vivo. FIG. 22A is a series of images of H&E-stained sections of H9 cells in Matrigel, H9 cells on a scaffold, progenitor on a scaffold, and engineered bone. FIG. 22B is a series of bar graphs and images showing quantitative histomorphometric analysis of staining intensity and % scaffold area cover using bone markers osteopontin, bone sialoprotein and osteocalcin in engineered bone compared to scaffolds seeded with H9-progenitors after 8 weeks in vivo. Data represent averages of 5 measurements±standard deviation (p<0.01; * represents a statistically significant difference between the groups). Insets represent negative staining controls.

FIG. 23 is a series of images showing additional examination of engineered bone tissue after explantation. FIG. 23, top row shows H&E staining of H9 progenitors on scaffold, H9-engineered bone, and high magnification images of H9-engineered bone. Arrows indicate the presence of vascularization. Asterisks indicate presence of osteoclastic cells. FIG. 23, bottom row shows human nuclear antigen expression in H9 progenitors on scaffold, H9-engineered bone, and negative control.

FIG. 24 is a series of images and a graph. FIG. 24A-C shows bone grafts engineered from human ESC. Mesenchymal-like progenitors were derived from ESC (FIG. 24A-osteogenic, FIG. 24B-chondrogenic, FIG. 24C-adipogenic potential, FIG. 24D-surface antigens of mesenchymal lineages), seeded into decellularized bone scaffolds (FIG. 24E) and cultured in perfusion bioreactors (FIG. 24E). Perfusion culture parameters were based on studies of BMSC and yielded grafts with significantly higher cellularity and bone-like extracellular matrix compared to static culture (FIG. 24F).

### DETAILED DESCRIPTION OF THE INVENTION

The present disclosure is based, at least in part, on the successful in vitro growth of living human bone using osteogenic progenitor cells derived from human embryonic stem cells (hESC) seeded in an osteoconductive scaffold of decellularized bone and perfused in a bioreactor culture. Described herein is a novel staged protocol to induce hESC differentiation; expand mesenchymal-like progenitor cells; seed and allow attachment of bone progenitor cells within the scaffolds; and support development of large viable pieces of bone by perfusing the cell-seeded constructs. Such a stepwise approach can avoid formation of tissues other than bone in the engineered construct.

Living bone, developed according to approaches described herein, can integrate and remodel following implantation, alleviating the problems of adhesive breakdown of current prosthetic devices and the need for their eventual replacement. Transplantation of bone tissue formed according to processes described herein, can provide healthy tissue function (e.g., mechanical support) and can enhance the process of regeneration. Composite tissue grafts developed according to processes disclosed herein can be used for, e.g., craniofacial and skeletal reconstructions. Such composite tissue grafts can also provide controllable models of high biological fidelity to study development and disease.

As shown herein, an in vitro model of bone development utilizing decellularized bovine bone scaffolds and a perfusion bioreactor can be applied to progenitor cells, such as ESC. Experiments described herein show that hESC-derived mesenchymal-like progenitors can form bone-like tissue under tissue engineering conditions for human mesenchymal cells from bone marrow (BMSC) (see e.g., Grayson W L, et al. Biotechnol Bioeng, 2011, 108(5):1159). Surprisingly, bone material produced as described herein can have reduced or substantially reduced capacity to induce abnormal tissue growth (e.g., teratoma) in a subject receiving engineered bone (see e.g., Example 5), in contrast to teratoma formation in animals receiving undifferentiated human embryonic stem cells in Matrigel or seeded in bone scaffolds. These results provide proof of principle support for development of clinical-size bone grafts from ESC, as well as models for advanced quantitative studies of bone development by recapitulating some aspects of native tissue in vitro.

Compositions and methods described herein can provide a supply of functional bone grafts (e.g., functional human bone grafts) for transplantation. Compositions and methods described herein can provide a platform for testing of agents (e.g., pharmaceutical or biopharmaceutical drugs) for their effect on bone formation. Approaches described herein can provide customized, patient-specific autologous bone grafts, with vascular compartment, as well as additional tissues, such as nerve muscle, or cartilage.

Progenitor Cells and Induction of Differentiation

In various embodiments, osteogenic progenitor cells are induced from less differentiated progenitor cells. For example, osteogenic progenitor cells can be induced from ESCs (e.g., hESC).

A progenitor cell, as that term is used herein, is a precursor to a osteogenic or osteogenic-like cell and can differentiate thereto. A progenitor cell can be a multipotent cell. A progenitor cell can be self-renewing. For example, a progenitor cell can be a mesenchymal stem cell (e.g., a human mesenchymal stem cell). The progenitor cell can be substantially less differentiated than a osteogenic or osteogenic-like cell. For example, a progenitor cell can be freshly isolated or not pre-treated with growth factors before being further cultured.

Progenitor cells can be isolated, purified, or cultured by a variety of means known to the art. Methods for the isolation and culture of tissue progenitor cells are discussed in, for example, Vunjak-Novakovic and Freshney (2006) Culture of Cells for Tissue Engineering, Wiley-Liss, ISBN 0471629359. Progenitor cells can be cultured in a differentiation medium. A differentiation medium can be any medium recognized in the art suitable to differentiate a progenitor cell into a cell capable of forming an osteogenic or osteogenic-like cell. For example, a differentiation medium can include one or more of DMEM, serum, dexamethasone, b-glycerophosphate, ascorbic acid, bone morphogenic protein, vitamin D, and pen-strep.

In various embodiments, a progenitor cell is a precursor to a osteogenic or osteogenic-like cell and can differentiate under culture conditions described herein. In some embodiments, a progenitor cell does not display a bone marker protein, such as collagen, osteopontin, and bone-sialoprotein. In some embodiments, a progenitor cell does not exhibit surface markers such as SSEA-1, SSEA-4, CD31, CD34, and CD271. In some embodiments, a progenitor cell does exhibit surface markers such as CD44, CD73, CD90, and CD166.

Progenitor cells can be derived from the same or different species as a transplant recipient. For example, progenitor cells can be derived from an animal, including, but not limited to, a horse, cow, dog, cat, sheep, pig, rabbit, goat, chicken, or human.

In some embodiments, the progenitor cells comprise human mesenchymal stem cells. In some embodiments, the progenitor cells comprise human embryonic stem cells. In some embodiments, the progenitor cells comprise bone marrow stromal/stem cells. In some embodiments, the progenitor cells comprise human induced pluripotent cell lines.

In some embodiments, ESCs are induced to differentiate into a mesenchymal-like progenitor cells. In some embodiments, the mesenchymal-like progenitor cells is expanded and the expanded cells used to seed a biocompatible scaffold.

Expansion of Progenitor Cells

Described herein are exemplary methods to induce osteogenic differentiation of progenitor on substrates, such as monolayer or 3D pellet culture of hMSCs (see Example 1-2). Progenitor cells can be cultured by a variety of means known to the art. Progenitor cells can be incubated under conditions allowing differentiation to osteogenic or osteogenic-like cells. Progenitor cells can be cultured by a variety of means known to the art. For example, progenitor cells can be plated (e.g., about 100,000 cells per well) for 2D culture. As another example, progenitor cells can be centrifuged (e.g., about 2 million cells) to form a 3D pellet. Monolayer (2D) or 3D cell pellets can be cultured in a suitable growth medium. Methods of culturing progenitor cells are generally known in the art and such methods can be adapted so as to provide optimal conditions for differentiation of progenitor cells contacted with CTGF or TGFβ3 (see e.g., Vunjak-Novakovic and Freshney (2006) Culture of Cells for Tissue Engineering, Wiley-Liss, ISBN 0471629359). Progenitor cells can be induced to differentiate in a first medium (e.g., a medium with serum and missing bFGF) and then expanded in a second medium (e.g., a medium with bFGF). Progenitor cells can be expanded on an expansion medium. An expansion medium can include bFGF. An expansion medium can include one or more of KO-DMEM, KO-serum replacement, serum, nonessential amino acids, glutamine, b-mercaptoethanol, or bFGF.

In some embodiments, a progenitor cell or an osteogenic or osteogenic-like cell can be co-cultured with one or more additional cell types. Such additional cell types can include (but are not limited to) blood cells, adipose cells, bone marrow cells, umbilical cord cells, cardiac cells, skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes.

Matrix and Scaffold

Various compositions and methods described herein employ a matrix. In some embodiments, progenitor cells are introduced into or onto the matrix so as to form a tissue module, such as a bone tissue module. In various embodiments, the matrix materials are formed into a 3-dimensional scaffold. The scaffold can contain one or more matrix layers.

The matrix or scaffold can: provide structural or functional features of the target tissue (e.g., bone); allow cell attachment and migration; deliver and retain cells and biochemical factors; enable diffusion of cell nutrients and expressed products; or exert certain mechanical and biological influences to modify the behavior of the cell phase. The matrix materials of various embodiments are biocompatible materials that generally form, for example, a porous, microcellular scaffold, or hydrogel or scaffold-gel composite, which provides a physical support and an adhesive substrate for introducing progenitor cells during in vitro fabrication or culturing and subsequent in vivo implantation.

A matrix with a high porosity and an adequate pore size can provide for increased cell introduction and diffusion throughout the whole structure of both cells and nutrients. Matrix biodegradability can provide for absorption of the matrix by the surrounding tissues (e.g., after differentiation and growth of bone tissues from progenitor cells) and can eliminate the necessity of a surgical removal. The rate at which degradation occurs should coincide as much as possible with the rate of tissue formation. Thus, while cells are fabricating their own natural structure around themselves, the matrix can provide structural integrity and eventually break down leaving the neotissue, newly formed tissue which can assume the mechanical load. Injectability is also preferred in some clinical applications. Suitable matrix materials are discussed in, for example, Ma and Elisseeff, ed. (2005) Scaffolding in Tissue Engineering, CRC, ISBN 1574445219; Saltzman (2004) Tissue Engineering: Engineering Principles for the Design of Replacement Organs and Tissues, Oxford ISBN 019514130X.

The matrix configuration can be dependent on the bone tissue that is to be produced. Preferably the matrix is a pliable, biocompatible, porous template that allows for target tissue growth. The matrix can be fabricated into structural supports, where the geometry of the structure is tailored to the application. The porosity of the matrix is a design parameter that influences cell introduction or cell infiltration. The matrix can be designed to incorporate extracellular matrix proteins that influence cell adhesion and migration in the matrix.

Matrices can include an osteoconductive scaffold, such as a native osteoconductive scaffold. Matrices can include decellularized bone. For example, The matrix can include a fully decellularized trabecular bone scaffold, for example a fully decellularized mammalian (e.g., human or bovine) trabecular bone scaffold.

Matrices can be produced from proteins (e.g. extracellular matrix proteins such as fibrin, collagen, and fibronectin), polymers (e.g., polyvinylpyrrolidone), polysaccharides (e.g. alginate), hyaluronic acid, or analogs, mixtures, combinations, and derivatives of the above.

The matrix can be formed of synthetic polymers. Such synthetic polymers include, but are not limited to, poly(ethylene) glycol, bioerodible polymers (e.g., poly(lactide), poly(glycolic acid), poly(lactide-co-glycolide), poly(caprolactone), polyester (e.g., poly-(L-lactic acid), polyanhydride, polyglactin, polyglycolic acid), polycarbonates, polyamides, polyanhydrides, polyamino acids, polyortho esters, polyacetals, polycyanoacrylates), polyphosphazene, degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, polyvinyl pyrrolidone, poly(vinylimidazole), chlorosulphonated polyolifins, polyethylene oxide, polyvinyl alcohol (e.g., polyvinyl alcohol sponge), synthetic marine adhesive proteins, Teflon®, nylon, or analogs, mixtures, combinations (e.g., polyethylene oxide-polypropylene glycol block copolymer; poly(D,L-lactide-co-glycolide) fiber matrix), and derivatives of the above.

The matrix can be formed of naturally occurring polymers or natively derived polymers. Such polymers include, but are not limited to, agarose, alginate (e.g., calcium alginate gel), fibrin, fibrinogen, fibronectin, collagen (e.g., a collagen gel), gelatin, hyaluronic acid, chitin, and other suitable polymers and biopolymers, or analogs, mixtures, combinations, and derivatives of the above. Also, the matrix can be formed from a mixture of naturally occurring biopolymers and synthetic polymers.

In some embodiments, one or more matrix materials are modified so as to increase biodegradability. For example, PCL is a biodegradable polyester by hydrolysis of its ester linkages in physiological conditions, and can be further modified with ring opening polymerization to increase its biodegradability.

Introduction of Cells to Matrix

To form various compositions described herein, progenitor cells can be introduced (e.g., implanted, injected, infused, or seeded) into or onto an artificial structure (e.g., a scaffold comprising a matrix material) capable of supporting three-dimensional tissue or organ formation. It is contemplated that more than one type of progenitor cell can be introduced into the matrix.

Progenitor cells can be introduced into the matrix material by a variety of means known to the art (see e.g., Example 3). Methods for the introduction (e.g., infusion, seeding, injection, hydrogel encapsulation within the scaffold, etc.) of progenitor cells into or into the matrix material are discussed in, for example, Ma and Elisseeff, ed. (2005) Scaffolding In Tissue Engineering, CRC, ISBN 1574445219; Saltzman (2004) Tissue Engineering: Engineering Principles for the Design of Replacement Organs and Tissues, Oxford ISBN 019514130X; Minuth et al. (2005) Tissue Engineering From Cell Biology to Artificial Organs, John Wiley & Sons, ISBN 3527311866. For example, progenitor cells can be introduced into or onto the matrix by methods including hydrating freeze-dried scaffolds with a cell suspension (e.g., at a concentration of 100 cells/ml to several million cells/ml).

Methods of culturing and differentiating progenitor cells in or on scaffolds are generally known in the art (see e.g., Saltzman (2004) Tissue Engineering: Engineering Principles for the Design of Replacement Organs and Tissues, Oxford ISBN 019514130X; Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). As will be appreciated by one skilled in the art, the time between progenitor cell introduction into or onto the matrix and engrafting the resulting matrix can vary according to particular application. Incubation (and subsequent replication or differentiation) of the engineered composition containing progenitor cells in or on the matrix material can be, for example, at least in part in vitro, substantially in vitro, at least in part in vivo, or substantially in vivo. Determination of optimal culture time is within the skill of the art.

A suitable medium can be used for in vitro progenitor cell infusion, differentiation, or cell transdifferentiation (see e.g., Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). For example, the culture medium can be an osteogenic culture medium. As another example, the culture medium can be an osteogenic culture medium including DMEM, FBS, beta-glycerophosphate, dexamethasone and ascorbate-2 phosphate.

The culture time can vary from about an hour, several hours, a day, several days, a week, or several weeks. The quantity and type of cells present in the matrix can be characterized by, for example, morphology by ELISA, by live cell staining, by protein assays, by genetic assays, by mechanical analysis, by RT-PCR, or by immunostaining to screen for cell-type-specific markers (see e.g., Minuth et al. (2005) Tissue Engineering From Cell Biology to Artificial Organs, John Wiley & Sons, ISBN 3527311866). For example, cell growth or osteogenesis can be determined according to live cell staining, DNA content, alkaline phosphatase activity or osteopontin release into culture medium. As another example, bone tissue formation can be assessed by H&E, Masson Trichrome, osteopontin, bone sialoprotein or osteocalcin stainings, or by μCT imaging.

The present teachings include methods for optimizing the density of progenitor cells (and their lineage derivatives) so as to maximize the regenerative outcome of a bone tissue. Cell densities in a matrix can be monitored over time and at end-points. Tissue properties can be determined, for example, using standard techniques known to skilled artisans, such as histology, structural analysis, immunohistochemistry, biochemical analysis, and mechanical properties. As will be recognized by one skilled in the art, the cell densities of progenitor cells can vary according to, for example, progenitor type, tissue or organ type, matrix material, matrix volume, infusion method, seeding pattern, culture medium, growth factors, incubation time, incubation conditions, and the like. Generally, for progenitor cells, the cell density in a matrix can be, independently, from 0.0001 million cells (M) ml⁻¹ to about 1000 M ml⁻¹. For example, the tissue progenitor cells and the vascular progenitor cells can each be present in the matrix at a density of about 0.001 M ml⁻¹, 0.01 M ml⁻¹, 0.1 M ml⁻¹, 1 M ml⁻¹, 5 M ml⁻¹, 10 M ml⁻¹, 15 M ml⁻¹, 20 M ml⁻¹, 25 M ml⁻¹, 30 M ml⁻¹, 35 M ml⁻¹, 40 M ml⁻¹, 45 M ml⁻¹, 50 M ml⁻¹, 55 M ml⁻¹, 60 M ml⁻¹, 65 M ml⁻¹, 70 M ml⁻¹, 75 M ml⁻¹, 80 M ml⁻¹, 85 M ml⁻¹, 90 M ml⁻¹, 95 M ml⁻¹, 100 M ml⁻¹, 200 M ml⁻¹, 300 M ml⁻¹, 400 M ml⁻¹, 500 M ml⁻¹, 600 M ml⁻¹, 700 M ml⁻¹, 800 M ml⁻¹, or 900 M ml⁻¹. In some embodiments, the cell density is about 30 M ml⁻¹ (see Example 3).

In some embodiments, a tissue module can comprise progenitor cells at a density of about 0.0001 million cells (M) ml⁻¹ to about 1000 M ml⁻¹. In some configurations, a tissue module can comprise progenitor cells at a density of at least about 1 M ml⁻¹ up to about 100 M ml⁻¹. In some configurations, a tissue module can comprise progenitor cells at a density of at least about 5 M ml⁻¹ up to about 95 M ml⁻¹. In some configurations, a tissue module can comprise progenitor cells at a density of at least about 10 M ml⁻¹ up to about 90 M ml⁻¹. In some configurations, a tissue module can comprise progenitor cells at a density of at least about 15 M ml⁻¹ up to about 85 M ml⁻¹. In some configurations, a tissue module can comprise progenitor cells at a density of at least about 20 M ml⁻¹ up to about 80 M ml⁻¹. In some configurations, a tissue module can comprise progenitor cells at a density of at least about 25 M ml⁻¹ up to about 75 M ml⁻¹. In some configurations, a tissue module can comprise progenitor cells at a density of at least about 30 M ml⁻¹ up to about 70 M ml⁻¹. In some configurations, a tissue module can comprise progenitor cells at a density of at least about 35 M ml⁻¹ up to about 65 M ml⁻¹. In some configurations, a tissue module can comprise progenitor cells at a density of at least about 40 M ml⁻¹ up to about 60 M ml⁻¹. In some configurations, a tissue module can comprise progenitor cells at a density of at least about 45 M ml⁻¹ up to about 55 M ml⁻¹. In some configurations, a tissue module can comprise progenitor cells at a density of at least about 45 M ml⁻¹ up to about 50 M ml⁻¹. In some configurations, a tissue module can comprise progenitor cells at a density of at least about 50 M ml⁻¹ up to about 55 M ml⁻¹.

In some embodiments, progenitor cells introduced to the matrix can comprise a heterologous nucleic acid so as to express a bioactive molecule such as heterologous protein, or to overexpress an endogenous protein. In non-limiting example, progenitor cells introduced to the matrix can express a fluorescent protein marker, such as GFP, EGFP, BFP, CFP, YFP, or RFP. In another example, progenitor cells introduced to the matrix can express an angiogenesis-related factor, such as activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors (e.g., various combinations of α subunits (e.g., α₁, α₂, α₃, α₄, α₅, α₆, α₇, α₈, α₉, α_{E}, α_{V}, α_{IIb}, α_{L}, α_{M}, α_{X}), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF164, VEGI, EG-VEGF, VEGF receptors, PF4, 16 kDa fragment of prolactin, prostaglandins E1 and E2, steroids, heparin, 1-butyryl glycerol (monobutyrin), or nicotinic amide. As another example, progenitor cells introduced to a matrix can comprise genetic sequences that reduce or eliminate an immune response in the host (e.g., by suppressing expression of cell surface antigens such as class I and class II histocompatibility antigen).

In some embodiments, one or more cell types in addition to progenitor cells can be introduced into or onto the matrix material. Such additional cell type can be selected from those discussed above, or can include (but not limited to) blood cells, adipose cells, bone marrow cells, umbilical cord cells, skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes. These cell-types can be introduced prior to, during, or after osteogenesis of the matrix. Such introduction may take place in vitro or in vivo. When the cells are introduced in vivo, the introduction may be at the site of the engineered bone tissue composition or at a site removed therefrom. Exemplary routes of administration of the cells include injection and surgical implantation.

Bioreactor and Perfusion of Constructs

In various embodiments, progenitor cells seeded in a scaffold are grown in vitro. For example, hESC seeded in a biocompatible scaffold can be grown in a perfusion bioreactor. Such an approach can provide increased cell survival, tissue formation, and bone matrix deposition, as compared to a static culture.

Suitable bioreactors and methods of their use are within the skill of the art (see e.g., Ma and Elisseeff, ed. (2005) Scaffolding in Tissue Engineering, CRC, ISBN 1574445219; Saltzman (2004) Tissue Engineering: Engineering Principles for the Design of Replacement Organs and Tissues, Oxford ISBN 019514130X; Haycock 2010 3D Cell Culture, Humana Press, 1^{st} Ed., ISBN-10: 1607619830).

The culture can be maintained, for example, in a bioreactor system, which may use a minipump for medium change. The minipump can be housed in an incubator, with fresh medium pumped to the matrix material of the scaffold. The medium circulated back to, and through, the matrix can have about 1% to about 100% fresh medium. The pump rate can be adjusted for optimal distribution of medium or additional agents included in the medium (see Example 4). The pump regime can be continuous, intermittent with constant or changing flow velocities, or combination of those. The medium delivery system can be tailored to the type of tissue or organ being manufactured. All culturing can be performed under sterile conditions.

Conditions for culturing progenitor cells in perfusion bioreactors to form bone tissue can be according to those described in Grayson W L, et al. Biotechnol Bioeng, 2011, 108(5):1159, incorporated herein by reference. For example, superficial flow velocities can be between about 80 and about 2,000 μm/s (e.g., about 80, about 100, about 200, about 300, about 400, about 500, about 600, about 700, about 800, about 900, about 1,000, about 1,100, about 1,200, about 1,300, about 1,400, about 1,500, about 1,600, about 1,700, about 1,800, about 1,900, or about 2,000 μm/s), corresponding to estimated initial shear stresses ranging from about 0.6 to about 20 mPa. As another example, superficial flow velocities can be between about 400 to about 800 μm/s. As another example, superficial flow velocities can be about 800 μm/s. Increased flow velocity has been shown to significantly affected cell morphology, cell-cell interactions, matrix production and composition, and the expression of osteogenic genes.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, preferably in purified form, together with a suitable amount of carrier so as to provide the form for proper administration to the subject.

The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Molecular Engineering

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem. Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Therapeutic Methods

Also provided is a process of treating a tissue defect, such as a bone tissue defect, in a subject in need. In some embodiments, a method of treatment of a bone tissue defect includes administration of a therapeutically effective amount of bone tissue composition or module as described herein.

Various embodiments provide a method of treating a tissue defect in a subject by implanting a tissue module described herein into a subject in need thereof. A determination of the need for treatment will typically be assessed by a history and physical exam consistent with the tissue defect at issue. A subject in need of the therapeutic methods described herein can be a subject having, diagnosed with, suspected of having, or at risk for developing a tissue defect. Subjects with an identified need of therapy include those with a diagnosed tissue defect. A determination of the need for treatment will typically be assessed by a history and physical exam consistent with the disease or condition at issue. Diagnosis of the various conditions treatable by the methods described herein is within the skill of the art. The subject is preferably an animal, including, but not limited to, mammals, reptiles, and avians, such as horse, cow, dog, cat, sheep, pig, rabbit, goat, chicken, or human.

As an example, a subject in need may have damage to a tissue, such as bone tissue, and the method provides an increase in biological function of the tissue by at least 5%, 10%, 25%, 50%, 75%, 90%, 100%, or 200%, or even by as much as 300%, 400%, or 500%. As yet another example, the subject in need may have a disease, disorder, or condition, and the method provides an engineered tissue module, e.g., an engineered bone tissue module, sufficient to ameliorate or stabilize the disease, disorder, or condition. For example, the subject may have a disease, disorder, or condition that results in the loss, atrophy, dysfunction, or death of bone tissue cells. Exemplary treated conditions include arthritis; osteoarthritis; osteoporosis; osteochondrosis; osteochondritis; osteogenesis imperfecta; osteomyelitis; osteophytes (i.e., bone spurs); achondroplasia; costochondritis; chondroma; chondrosarcoma; herniated disk; Klippel-Feil syndrome; osteitis deformans; osteitis fibrosa cystica, a congenital defect that results in the absence of a tissue; accidental tissue defect or damage such as fracture, wound, or joint trauma; an autoimmune disorder; diabetes (e.g., Charcot foot); cancer; a disease, disorder, or condition that requires the removal of a tissue (e.g., tumor resection); periodontal disease; and implant extraction. In a further example, the subject in need may have an increased risk of developing a disease, disorder, or condition that is delayed or prevented by the method.

Implantation of a tissue module, such as a bone tissue module, described herein is within the skill of the art. The matrix or cellular assembly can be either fully or partially implanted into a tissue or organ of the subject to become a functioning part thereof. In some embodiments, the implant initially attaches to and communicates with the host through a cellular monolayer. In some embodiments, over time, the introduced cells can expand and migrate out of the polymeric matrix to the surrounding tissue. After implantation, cells surrounding the tissue module can enter through cell migration. The cells surrounding the tissue module can be attracted by biologically active materials, including biological response modifiers, such as polysaccharides, liposomes, lipid vesicles with biologicals, proteins, peptides, genes, antigens, and antibodies which can be selectively incorporated into the matrix to provide the needed selectivity, for example, to tether the cell receptors to the matrix or stimulate cell migration into the matrix, or both. Generally, the matrix is porous, allowing for cell migration, augmented by both biological and physical-chemical gradients. For example, cells surrounding the implanted matrix can be attracted by biologically active materials including one or more of VEGF, fibroblast growth factor, transforming growth factor-beta, endothelial cell growth factor, P-selectin, and intercellular adhesion molecule. One of skill in the art will recognize and know how to use other biologically active materials that are appropriate for attracting cells to the matrix.

The methods, compositions, and devices of the application can include concurrent or sequential treatment with one or more of enzymes, ions, growth factors, and biologic agents, such as thrombin and calcium, or combinations thereof. The methods, compositions, and devices of the application can include concurrent or sequential treatment with non-biologic or biologic drugs.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to progenitor cells, culture media, and matrix or scaffold materials, as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

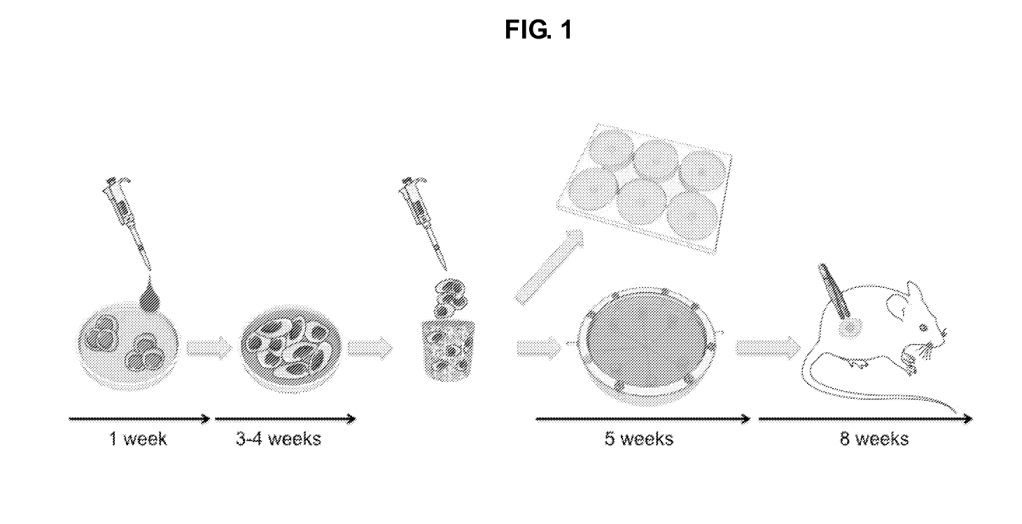

This example provides for an in vivo tissue engineering model. Analogous with previous work using stepwise differentiation to derive chondrogenic, cardiovascular and other lineages (Oldershaw, 2010; Yang, 2008; Murry, 2008) and to avoid formation of tissues other than bone, a stepwise protocol to engineer bone-like constructs from hESC was developed (see e.g., FIG. 1 for a general schematic). First, the undifferentiated hESC were induced into mesenchymal-like progenitors, selected and expanded in monolayer culture. Scaffold seeding was performed according to the following.

Bone scaffolds were prepared as in a previously published study (Grayson et al., Tiss Eng 2008).

For seeding to osteoconductive scaffolds, the progenitors hESC-P were cultured to P4 (passage 4), trypsinized, and seeded in suspension at a density of 30 million cells/ml. Uniform seeding was achieved by flipping the scaffolds every 20 minutes (3-times), followed by a 3 day incubation period in osteogenic medium in static culture.

ESC-progenitors and BMSC seeded on fully decellularized bovine trabecular bone scaffolds (4 mm Ø×4 mm) were cultured in osteogenic medium (DMEM with 10% FBS, beta-glycerophosphate, dexamethasone and ascorbate-2 phosphate), statically or in perfusion bioreactors providing interstitial flow through the developing tissues, for a period of time (5 weeks unless otherwise noted). Interstitial velocity of 800 μm/sec was selected based on previous studies with BMSC (Grayson W L, et al. Biotechnol Bioeng, 2011).

The perfusion protocol resulted in uniform distribution of cells throughout the scaffolds.

### Example 2

The following example shows the induction of human embryonic stem cells (hESC) differentiation and derivation of mesenchymal progenitors. Undifferentiated cultures of hESC give rise to fibroblastic cells with resemblance to mesenchymal stem cells. Induced differentiation of hESC (lines H9 and H13) was completed by supplementing medium with serum and eliminating bFGF for 7 days, after which the cultures were split and passaged.

Mesenchymal-like progenitor (hESC-P) expansion was accomplished by means of expanding pluripotent hESC (lines H9, H13, Wicell Research Institute) on mitomycin-C inactivated mouse embryonic fibroblasts in expansion medium (80% KO-DMEM, 20% KO-serum replacement, 1% nonessential amino acids, 1 mM L-glutamine, 0.1 mM b-mercaptoethanol, 5 ng/ml bFGF 5, all from Invitrogen) and by expanding derived hESC-P cells. More specifically, hESC were split using collagenase digestion every 3-5 days, and monitored for typical hESC morphology as well as mechanically cleaned of differentiating cells.

For induction, hESC (P31-P43) were cultured until confluence for up to 7 days, when the medium was switched to mesoderm induction medium (same as expansion medium without bFGF, and with replacing KnockOut-serum with 20% fetal bovine serum) for 7 days. Following induction, resulting cells were split using trypsin-EDTA and seeded at 100,000 cells/cm² to gelatin coated tissue-culture dishes, followed by subculture. More specifically, adherent cells from induced hESC cultures were grown to confluence and subcultured using trypsin-EDTA for minimum of 3 passages, when uniform spindle-shaped morphology was observed. BMSC (Lonza) were used as controls.

### Example 3

This example shows various methods of cell characterization including measurement of surface antigens, histology staining analyses to determine potential for osteogenic, chondrogenic, and adipogenic differentiation.

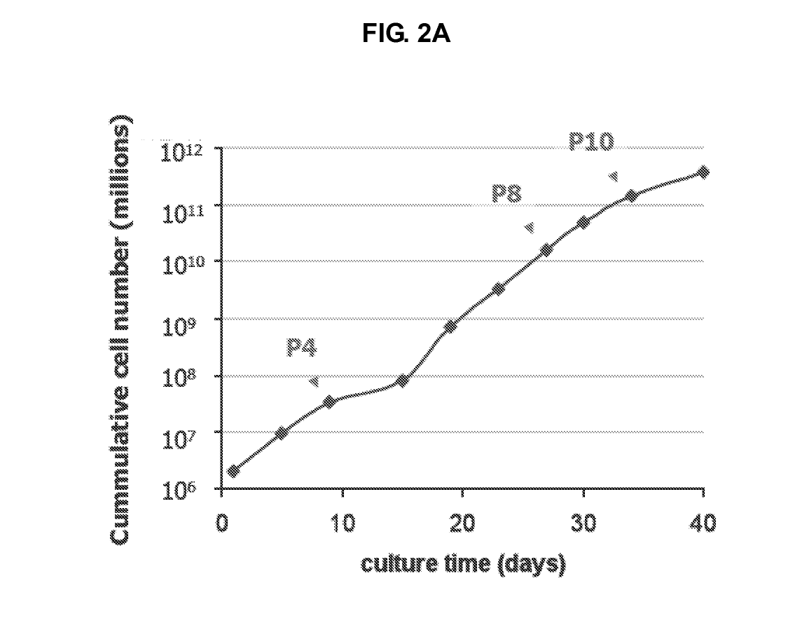

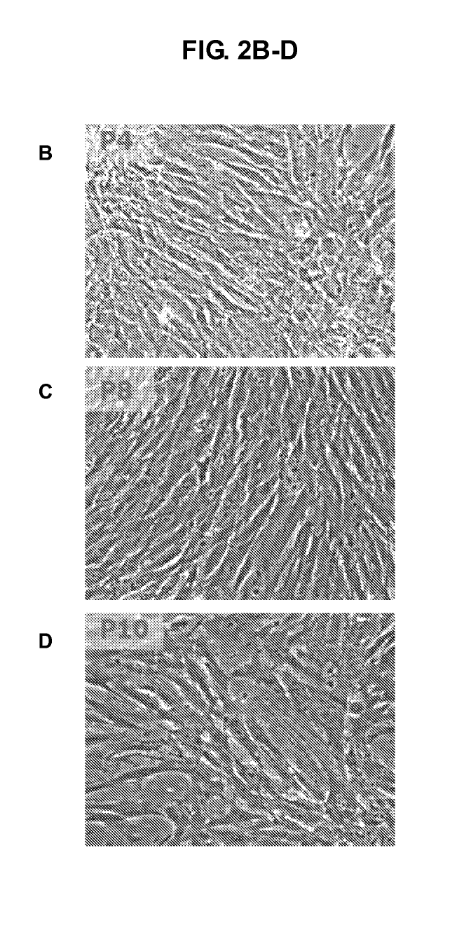

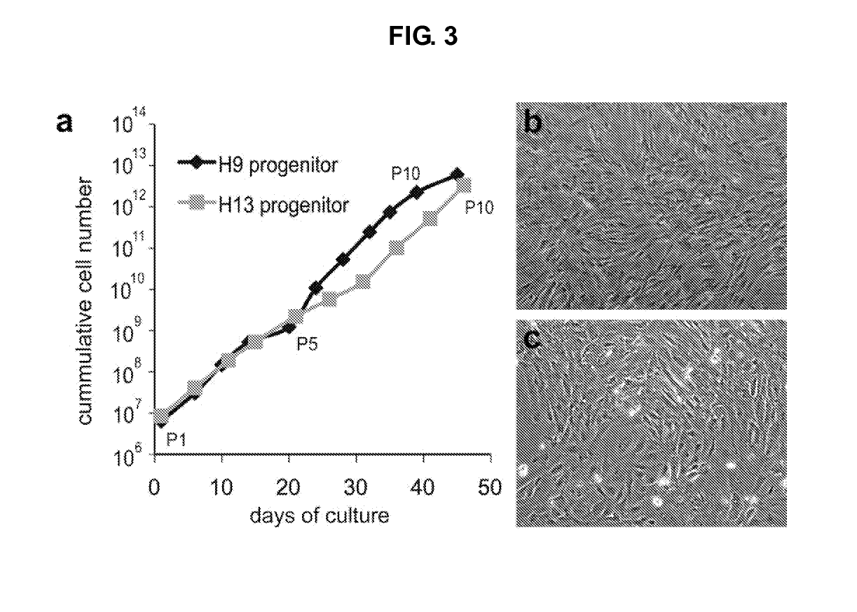

Growth and morphology of ESC-derived progenitors was also shown. Adherent progenitors exhibited continuous growth, derived from H9 and H13 lines (see e.g., FIG. 2A (H9) and FIG. 3A (H13)) and developed fibroblastic morphology (see e.g., FIGS. 2B-D and FIGS. 3B-C).

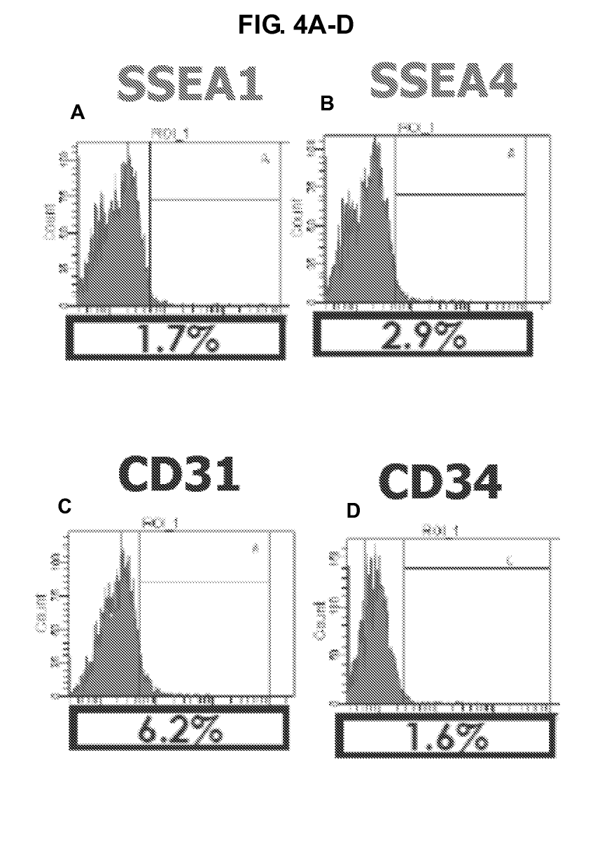

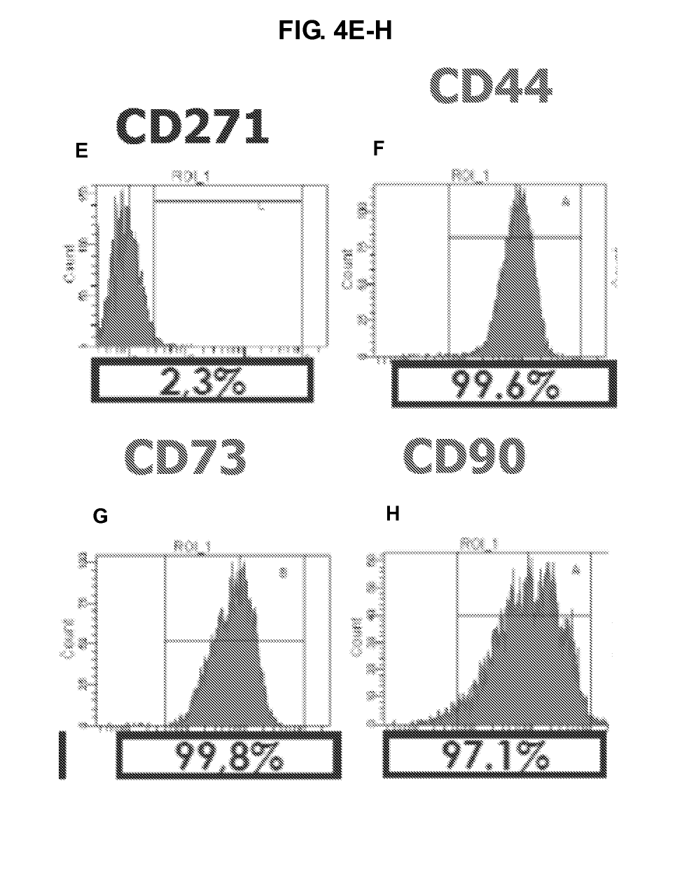

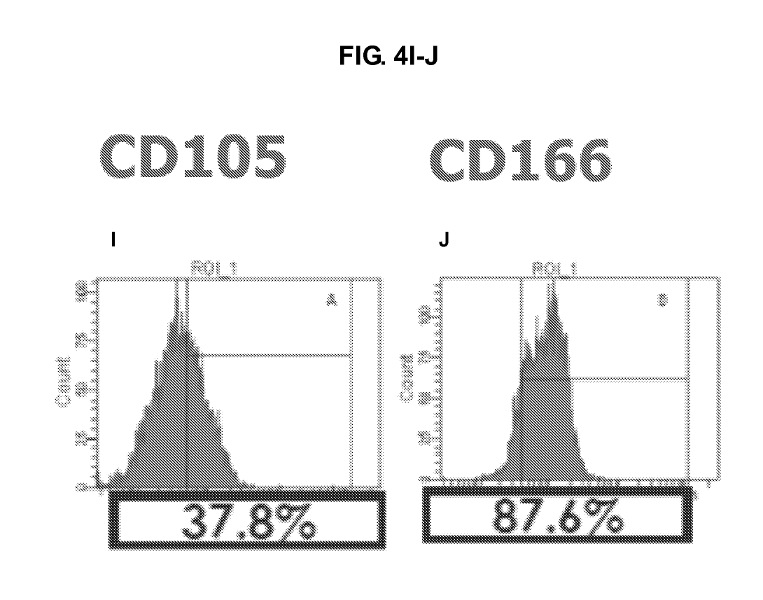

Surface antigen expression patterns of hESC-progenitors and BMSCs were determined. Analysis of surface markers were performed between P1-P9 and exhibited a uniform profile of negative (SSEA-1, SSEA-4, CD31, CD34, CD271) and positive (CD44, CD73, CD90, CD166) expression of surface markers (see e.g., FIG. 4A-J for H9 progenitors and Table 1 for H9 and H13 progenitors). A homogenous population developed between passages 1 and 3 expressing surface antigens CD44, CD73, CD90, CD105, CD166 that are typically found on mesenchymal cells (see e.g., Table 1). Expression of antigens marking pluripotent stem cells (SSEA-4), early differentiation of pluripotent stem cells (SSEA-1) and endothelial- (CD31), hematopoietic- (CD34) and neuroectodermal (CD271) lineages was negative (see e.g., Table 1). Surface antigen expression patterns of H9 and H13 hESC-progenitors and BMSC were measured (see e.g., Table 1). Expression was designated positive (+) when 70% or more of the population expressed the specific marker (e.g., surface antigen); expression was designated weakly positive (+−) when 20-70% of the population expressed the specific marker; expression was designated negative (−) when less than 20% of the population expressed the specific marker.

| Surface | H9-progenitor | | | | | H13-progenitor | | | | |
|---|---|---|---|---|---|---|---|---|---|---|
| antigen | P1 | P3 | P5 | P7 | P10 | P1 | P3 | P5 | P7 | P10 |
| SSEA-1 | − | − | − | − | − | − | − | − | − | − |
| SSEA-4 | − | − | − | − | − | − | − | − | − | − |
| CD31 | − | − | − | − | − | − | − | +− | − | − |
| CD34 | − | − | − | − | − | − | − | − | − | − |
| CD44 | + | + | + | + | + | + | + | + | + | + |
| CD73 | + | + | + | + | + | +− | + | + | + | + |
| CD90 | + | + | + | + | + | + | + | + | + | + |
| CD105 | − | +− | − | +− | − | − | − | +− | + | + |
| CD166 | + | + | + | + | + | +− | + | + | + | + |
| CD271 | − | − | − | − | − | − | − | − | − | − |

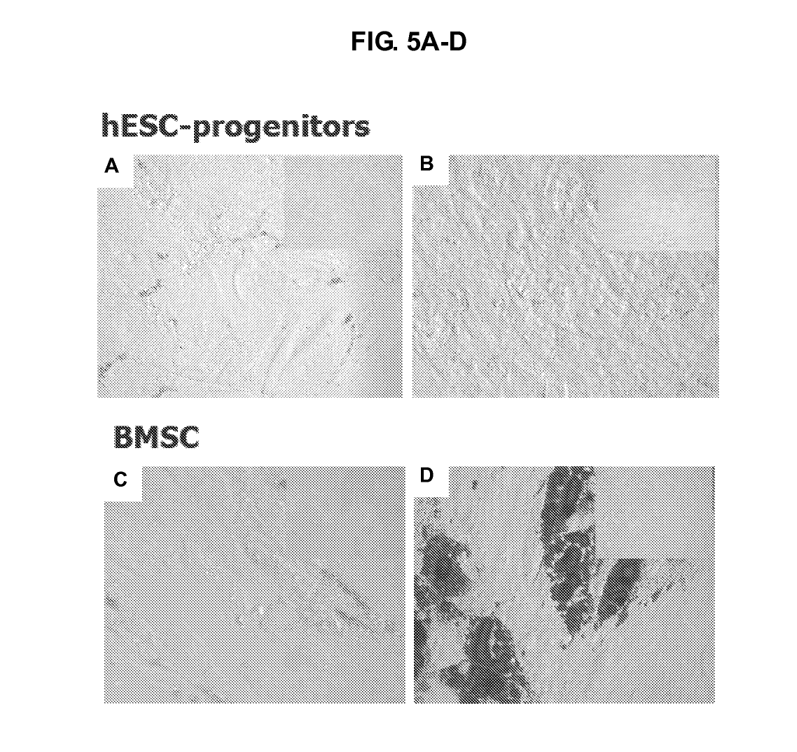

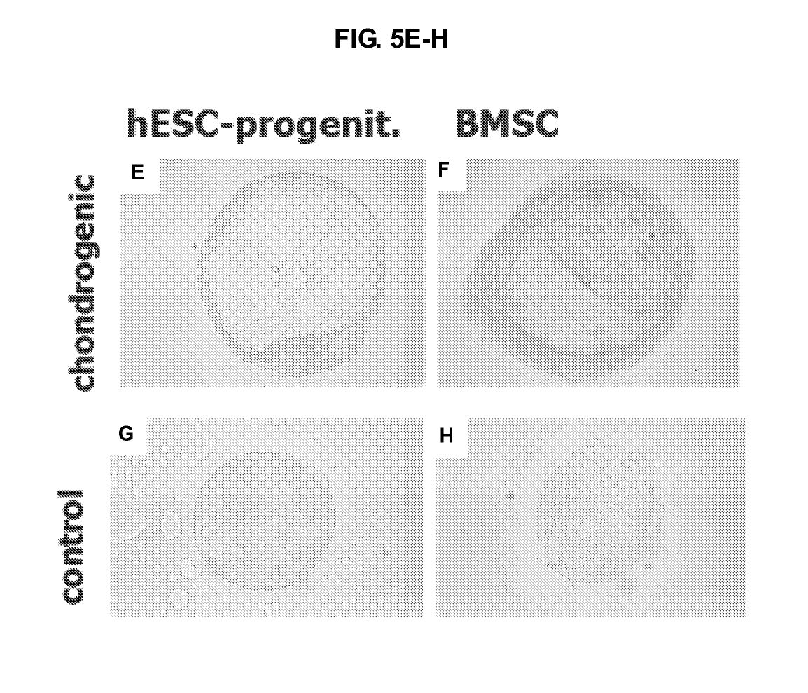

Both ESC lines were differentiated into progenitors that proliferated steadily over 10-11 passages, expressed mesenchymal surface antigens (>85% positive for CD44, CD73, CD90, CD166) (see e.g., Table 1) and exhibited strong osteogenic (AP activity, matrix mineralization), and weak chondrogenic (GAG deposition) and adipogenic (lipid vacuoles deposition) potentials (see e.g., FIG. 5).

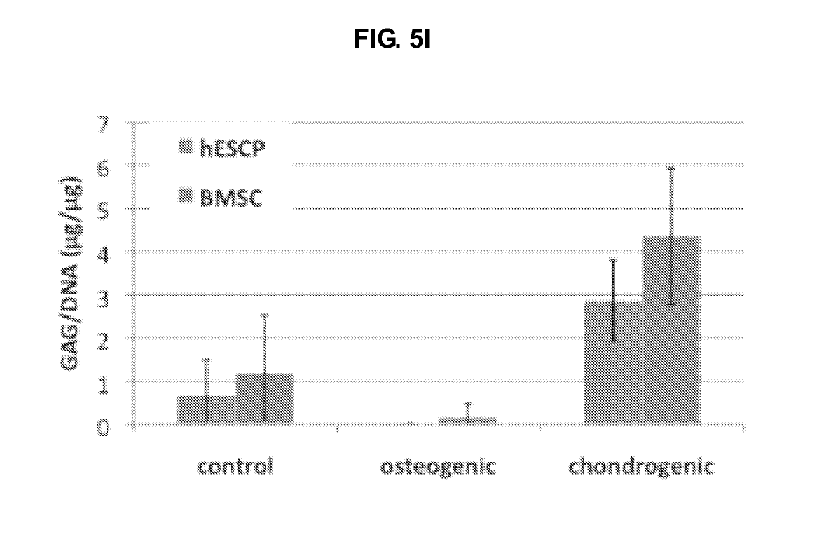

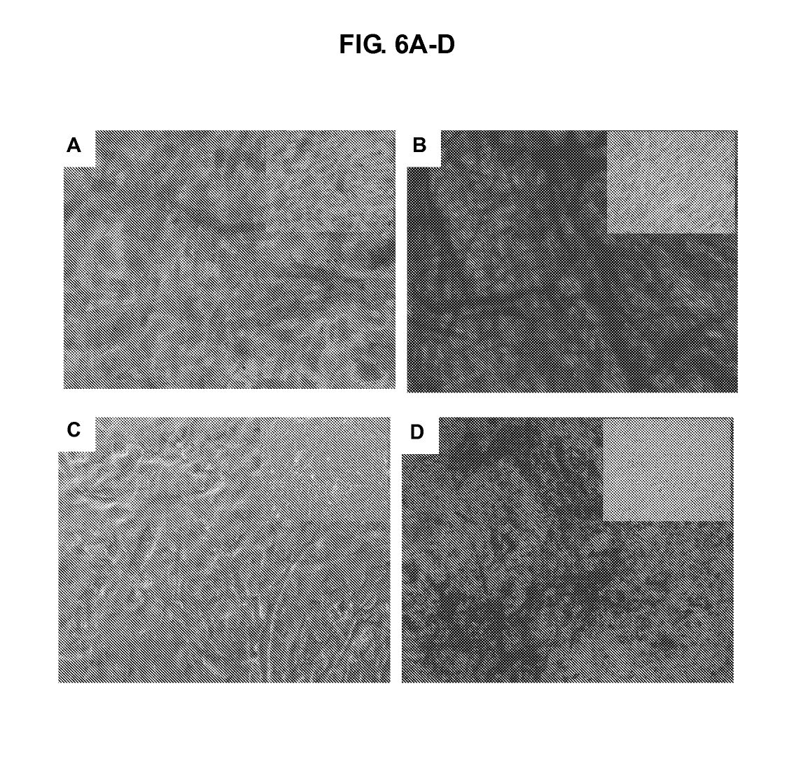

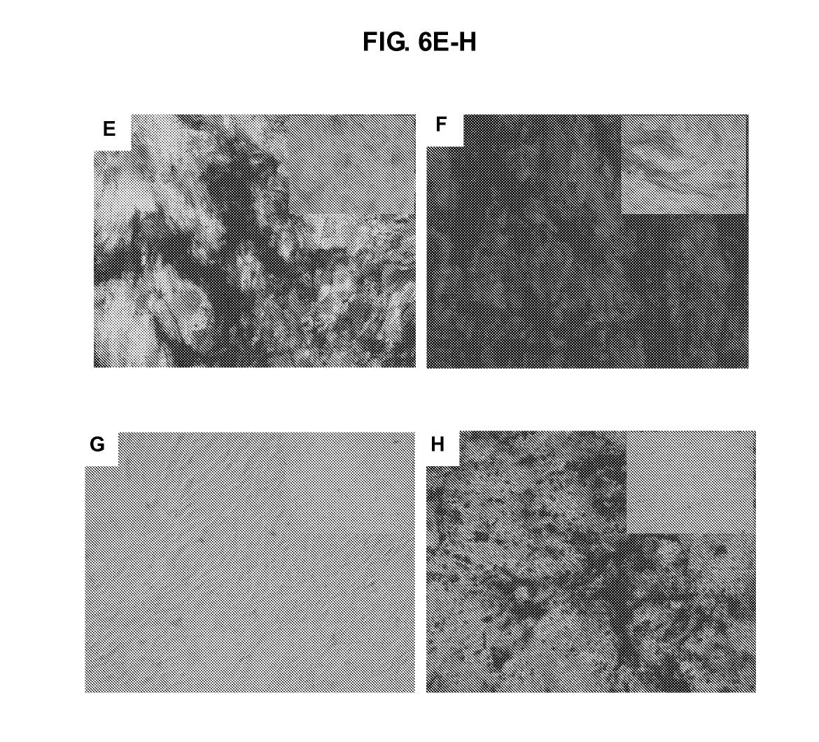

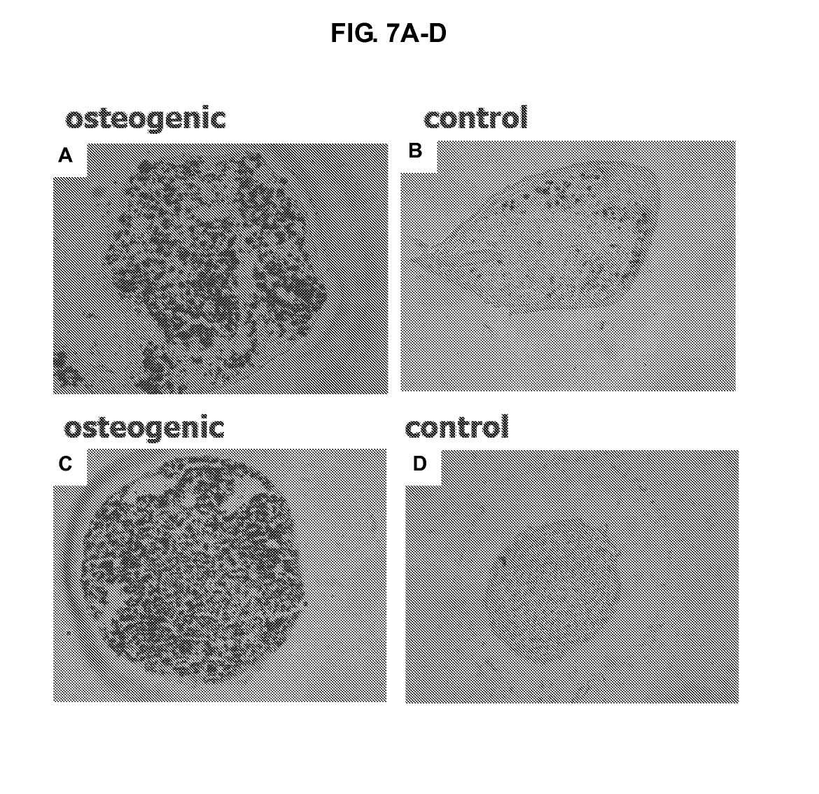

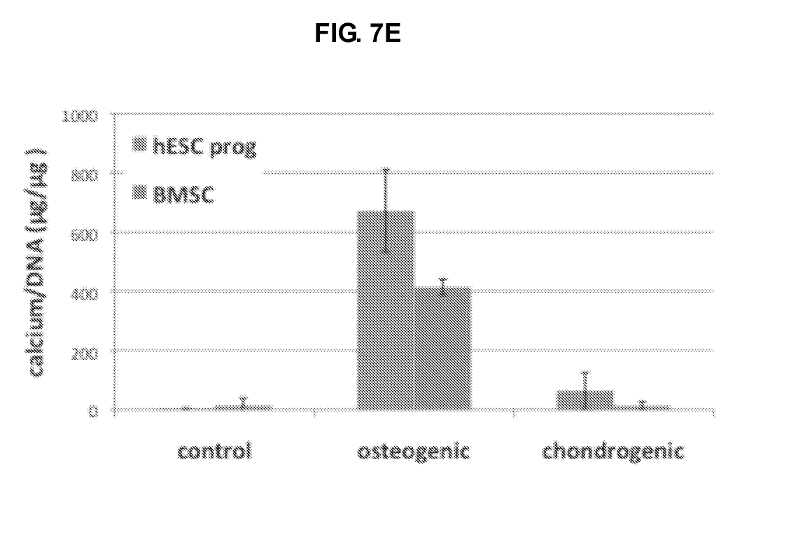

Potential for osteogenic and adipogenic differentiation was verified in monolayer cultures. H9 hESC progenitors were induced with osteogenic medium (DMEM, 10% fetal bovine serum, 1 μM dexamethasone, 10 mM b-glycerophosphate, 50 uM ascorbic acid 2-phosphate, 1% pen-strep) and adipogenic medium (induction: DMEM, 10% fetal bovine serum, 1 μM dexamethasone, 10 μg/ml insulin, 200 μM indomethacin, 500 μM IBMX, 1% pen-strep; maintenance: DMEM, 10% fetal bovine serum, 10 μg/ml insulin, 1% pen-strep) and evaluated between weeks 1-4 by alkaline phosphatase (AP) activity stain (see e.g., FIGS. 6A, B, E, and F, von Kossa stain for mineralization (calcification) (see e.g., FIGS. 6C, D, G, and H) and oil red-0 stain (see e.g., FIGS. 5A-B) for lipid accumulation. Potential for chondrogenic and osteogenic differentiation was checked in pellet cultures (see e.g., FIG. 7). hESC progenitors were induced with osteogenic medium and chondrogenic medium (DMEM, 100 nM dexamethasone, 50 μg/ml ascorbic acid 2-phosphate, 40 μg/ml L-proline, 1×ITS supplement, 1 mM sodium pyruvate, 10 ng/ml tumor growth factor-b3, 1% pen-strep) and evaluated on week 4 by histology (von Kossa stain, Alcian-blue stain for glycosaminoglycans (GAGs)) and biochemistry (DNA, glycosaminoglycans and calcium content). hESC H9 progenitors exhibited strong osteogenic and weak adipogenic and chondrogenic potential in these assays compared to control (bone marrow stromal/stem cells, BMSC) (see e.g., FIGS. 7A-E and FIGS. 5A-I).

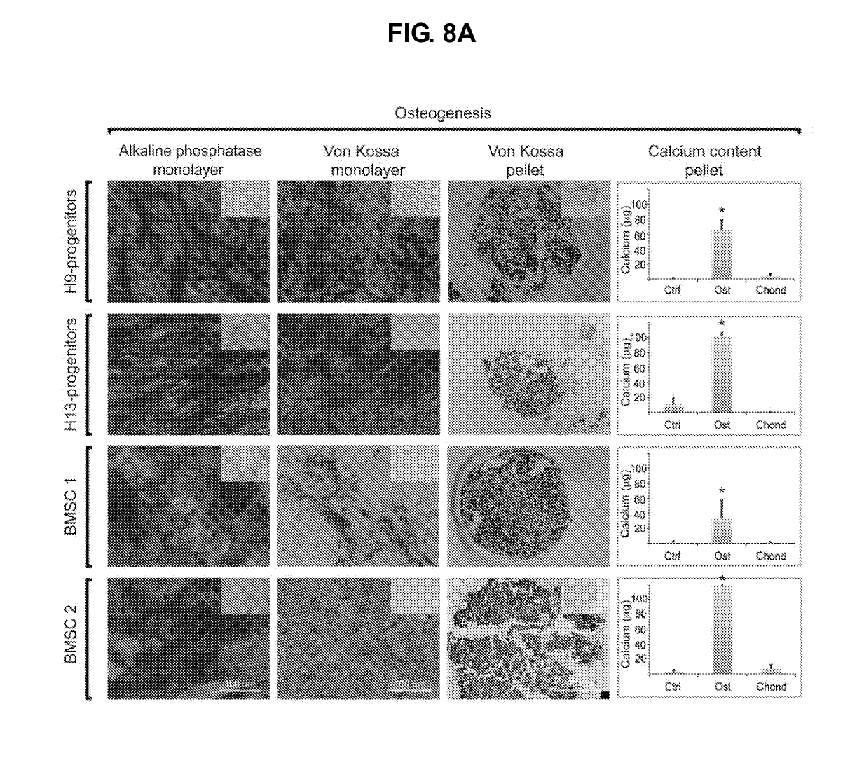

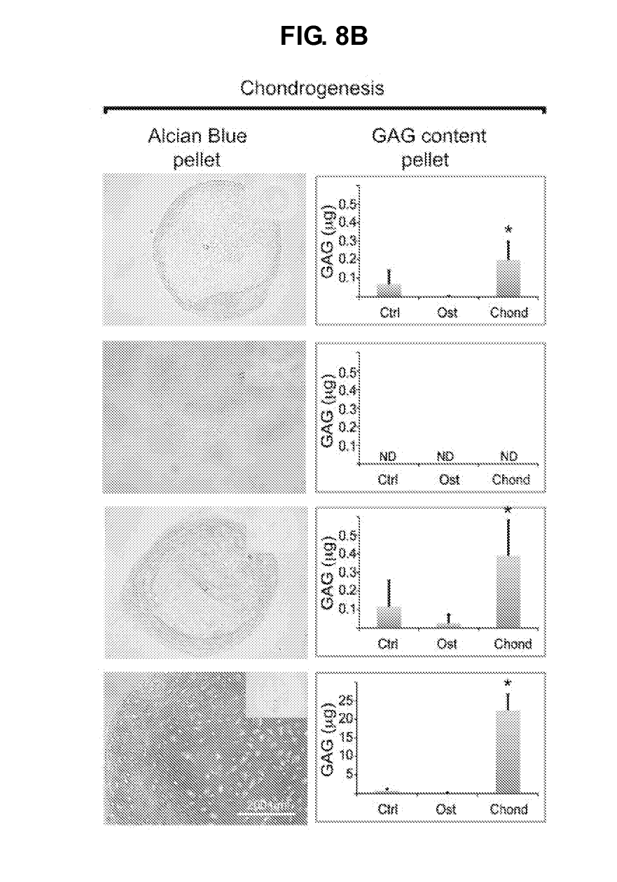

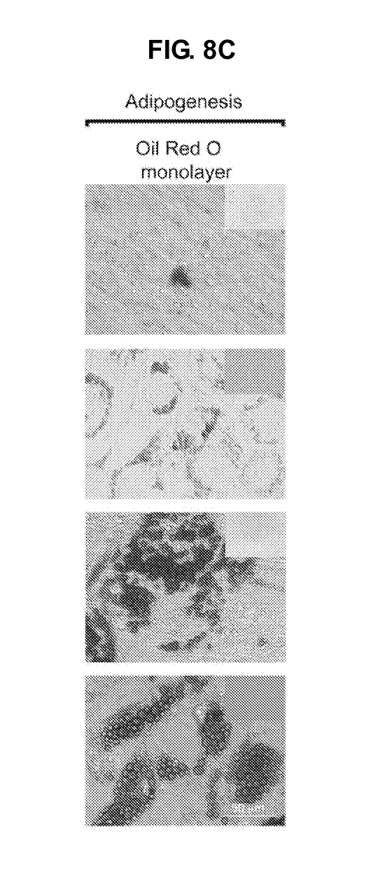

This example further shows mesenchymal differentiation potential of ESC-progenitors and BMSC. H9- and H13-progenitor potential for osteogenesis was evidenced by positive staining of alkaline phosphatase activity (purple) in monolayer cultures, and by matrix mineralization (von Kossa staining—black) in monolayer and pellet cultures stimulated with osteogenic medium (insets—cultures in control medium). Mineralization was confirmed by biochemical evaluation of the pellet calcium content, which was significantly increased in osteogenic medium (Ost) compared to control (Ctrl) and chondrogenic (Chond) media. Osteogenesis of ESC-derived progenitors was comparable to that of BMSC (see e.g., FIG. 8A). Weak chondrogenic potential was detected in H9-progenitor pellets compared to BMSC pellets by positive staining of glycosaminoglycans (Alcian Blue) and by determination of glycosaminoglycans content, which was significantly increased in chondrogenic medium (see e.g., FIG. 8B). Similarly, weak adipogenic potential of ESC-progenitors compared to BMSC was observed by positive staining of lipid vacuoles (Oil Red O) in monolayer cultures stimulated with adipogenic medium (see e.g., FIG. 8C).

H9- and H13-derived mesenchymal progenitors show differentiation into osteogenic, chondrogenic and adipogenic lineages (see e.g., FIG. 8). The osteogenic potential of these cells was very strong and comparable to that of BMSCs, as shown by alkaline phosphatase activity and matrix mineralization in osteogenic medium (see e.g., FIG. 8). In contrast, the chondrogenic potential, measured by Alcian Blue staining and glycosaminoglycans content in cells cultured in chondrogenic medium and adipogenic potential of cells cultured in adipogenic medium were relatively weaker.

### Example 4

This example shows the robust development of bone matrix in perfusion culture of hESC-progenitors in bone scaffolds. Previous studies of BMSC (Grayson, 2010; Grayson, 2011) and ASC (Frohlich, 2010) demonstrated the potential of decellularized bone scaffolds to support mesenchymal cell proliferation and bone-like tissue deposition in perfusion. Here, it is shown that the same culture model and operation parameters optimized for BMSCs (Grayson, 2011) will support the survival, growth and bone tissue formation by H9- and H13-derived mesenchymal progenitors.

Bone development in vitro was determined by the use of cell-seeded bone scaffolds cultured in a round bioreactor that provided controllable perfusion of culture medium through 6 engineered bone discs. The bioreactor was according to Grayson et al., Tiss Eng 2008. Bone tissue constructs were cultured for 5 weeks with perfusion of osteogenic medium. Constructs were kept at constant linear perfusion velocity of 800 μm/s. One half of the medium in the bioreactor (20 ml) was changed twice per week. Bone tissue development was evaluated at weeks 3 and 5, and compared to control static cultures that were prepared in parallel.

Biochemical analyses demonstrated increased DNA content in perfused constructs as compared to control cultures. Histological analyses demonstrated extensive formation of bone tissue as compared to control cultures. Immunohistochemical analyses indicated the formation of tissue composed of bone marker proteins (collagen, osteopontin, bone-sialoprotein). Micro-computerized tomography (μCT) analyses demonstrated progressive increase of mineralized tissue volume. Notably, in static cultures bone tissue formation was restricted to the outer surfaces of the constructs.

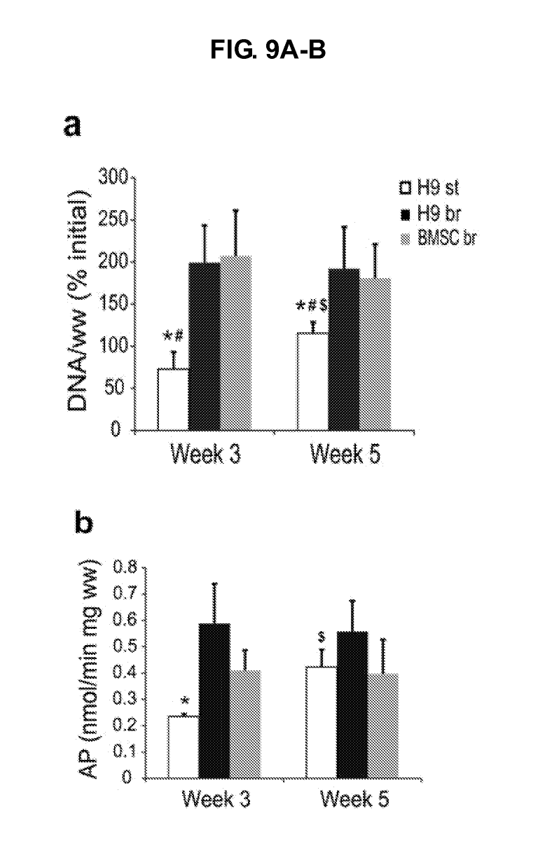

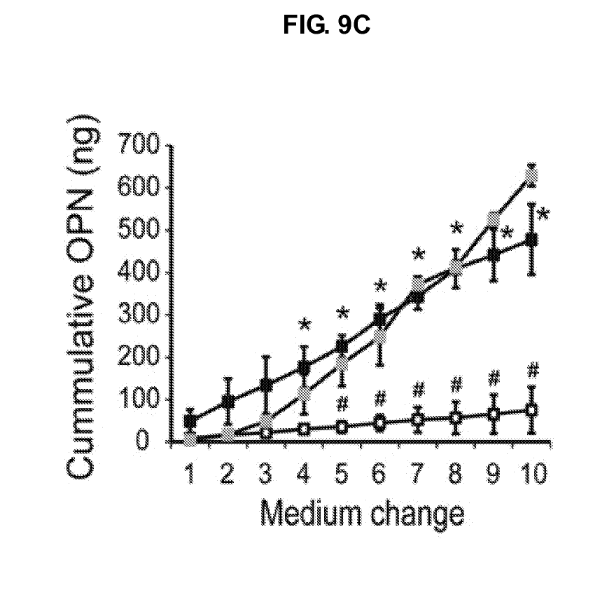

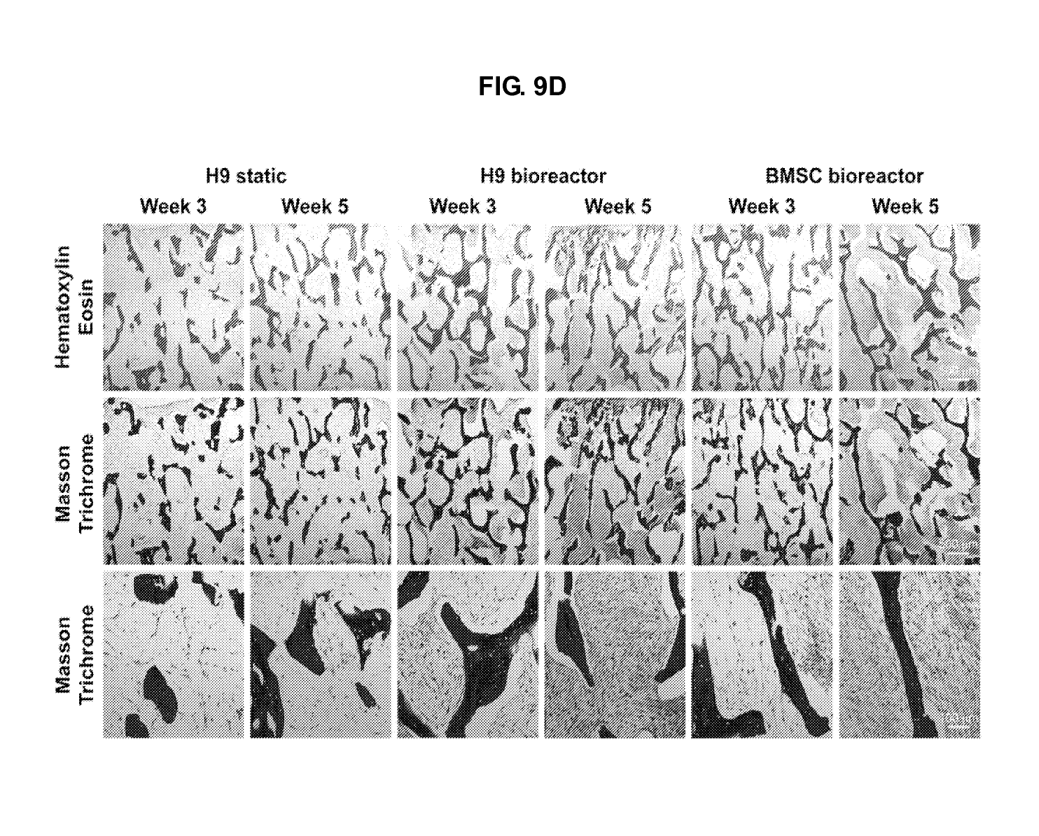

Bioreactor culture yielded constructs with significantly higher cellularity, AP activity, and osteopontin release into culture medium as compared to static cultures. Positive effects of bioreactor culture were determined by measuring DNA content per wet weight (ww) of tissue constructs (expressed as percent initial value at the start of bioreactor/static cultivation). Significantly higher DNA ww values were observed for the bioreactor group compared to the static group (see e.g., FIG. 9A). Similarly, positive effects of bioreactor culture were determined by measuring AP.

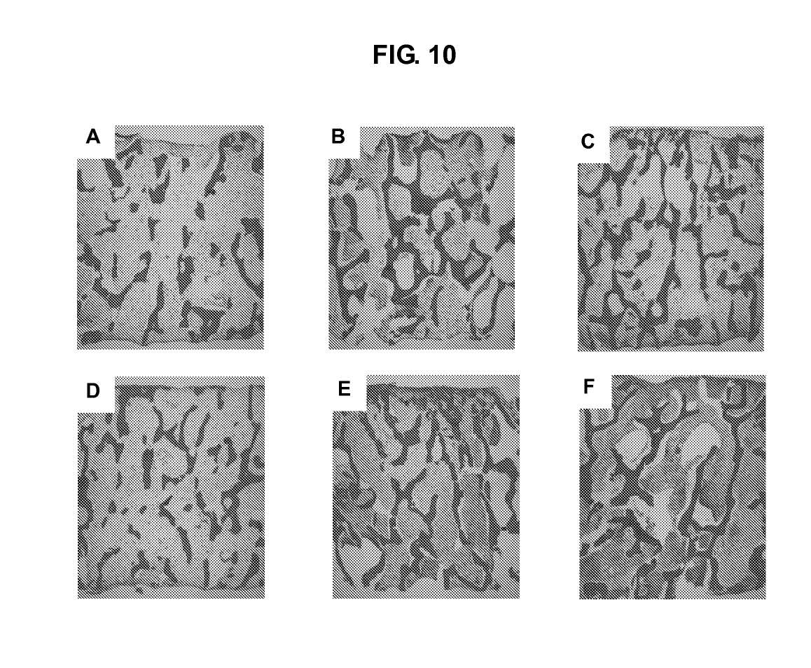

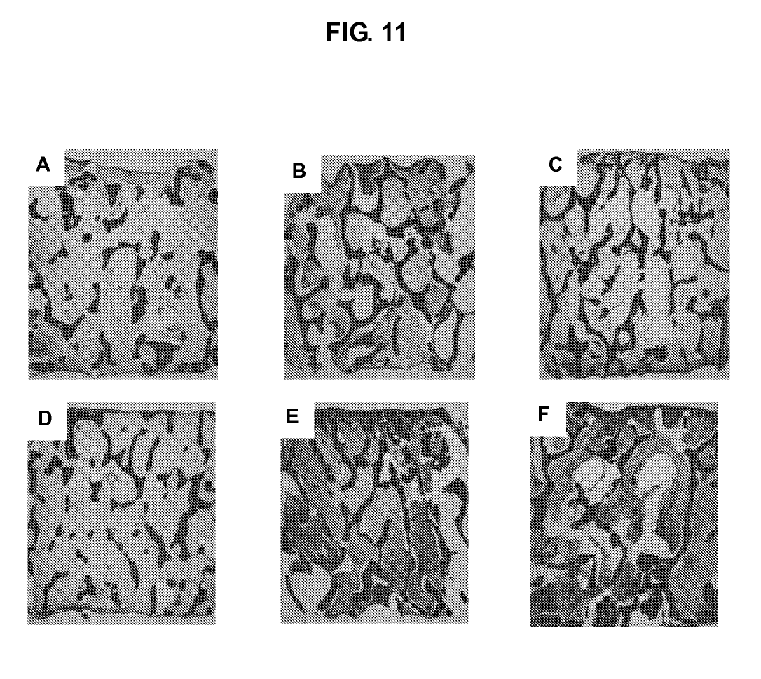

Significantly higher cell numbers for the br (perfused) group (see e.g., FIG. 9A), alkaline phosphatase activity (see e.g., FIG. 9B) and osteopontin release (see e.g., FIG. 9C) for constructs cultured in perfusion compared to static cultures was observed. Both DNA content and AP activity increased significantly in the static group from week 3 to week 5. Cumulative OPN was measures with respect to medium change. Cumulative osteopontin (OPN) release into culture medium was observed to be significantly higher after 2 weeks of culture (medium change 4 and later) in the bioreactor groups compared to static group (see e.g., FIG. 9C). Positive effects of bioreactor culture are confirmed by histological analyses (H&E), showing denser tissue deposition in the bioreactor groups compared to the static group after 5 weeks of culture (see e.g., FIG. 9D and FIG. 10). Masson Trichrome staining indicated deposition of collagenous matrix (blue color) in the bioreactor groups (see e.g., FIG. 9D and FIG. 11). Moreover, the enhancements in the biochemical compositions of the engineered bone matrix in perfusion vs. static culture were comparable for hESC and bone marrow derived mesenchymal progenitors (see e.g., FIG. 9).

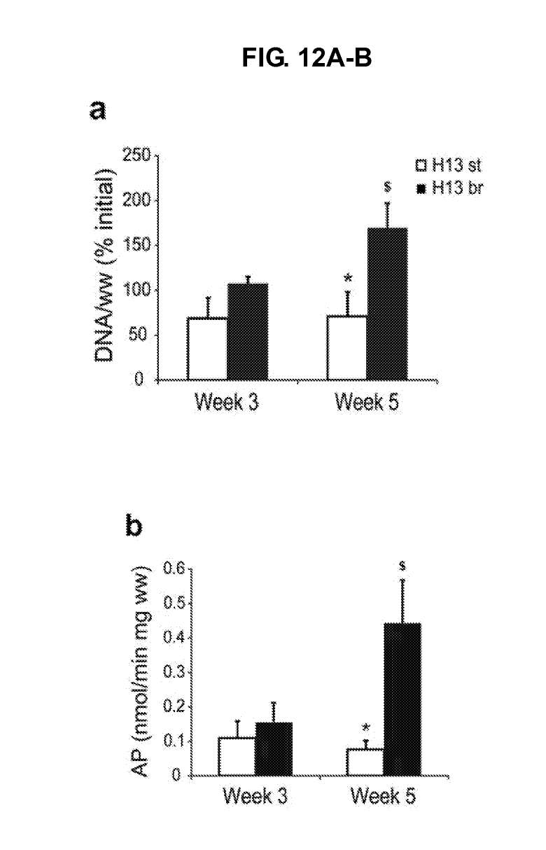

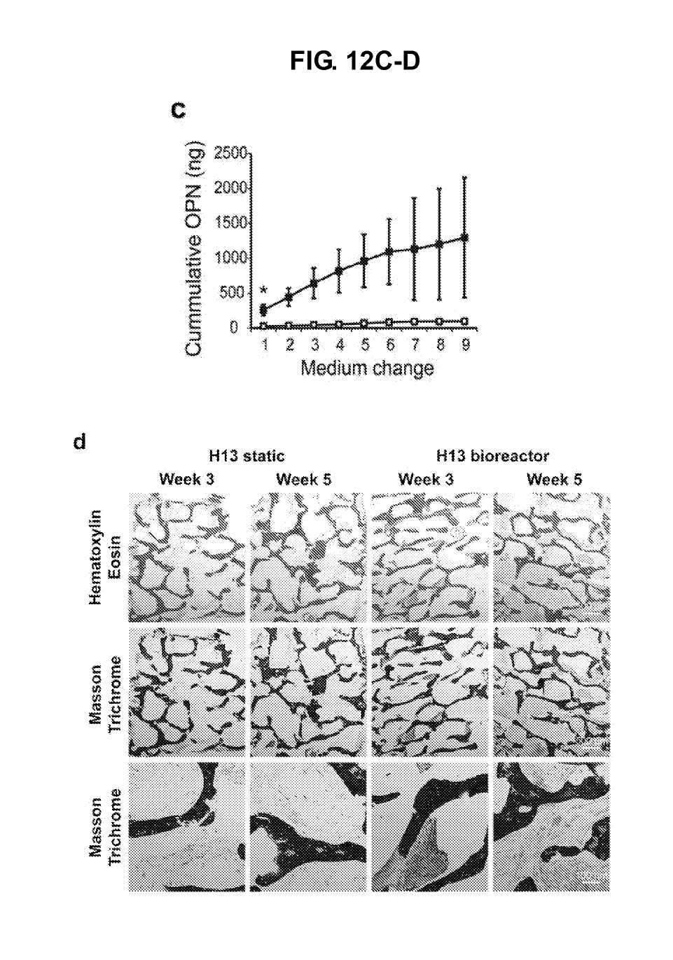

The following further shows the effect of bioreactor cultivation on tissue development of H13 progenitors. Both DNA content per ww (see e.g., FIG. 12A) and AP activity (see e.g., FIG. 12B) significantly increased in the bioreactor group from week 3 to week 5 of culture, and were found to be significantly higher compared to the static group after 5 weeks of culture. Cumulative osteopontin (OPN) release into culture medium was significantly higher during the first week of culture (medium change 1), and remained high compared to the static group throughout the culture (see e.g., FIG. 12C). Positive effects of bioreactor culture were also corroborated by histological analyses (see e.g., FIG. 12D), showing denser tissue deposition in the bioreactor group and the presence of collagenous matrix (positive Masson Trichrome staining).

After 3 weeks of culture, H13 progenitor constructs exhibited slightly lower cellularity and more variation in tissue density and distribution than H9 cells, suggesting differences in attachment or growth pattern between the cell lines (see e.g., FIG. 9 and FIG. 12). However, significant enhancements of tissue development for all 3 cell sources at the end of 5 weeks of culture in a bioreactor indicated the vital role of interstitial medium flow (see e.g., FIG. 9 and FIG. 12).

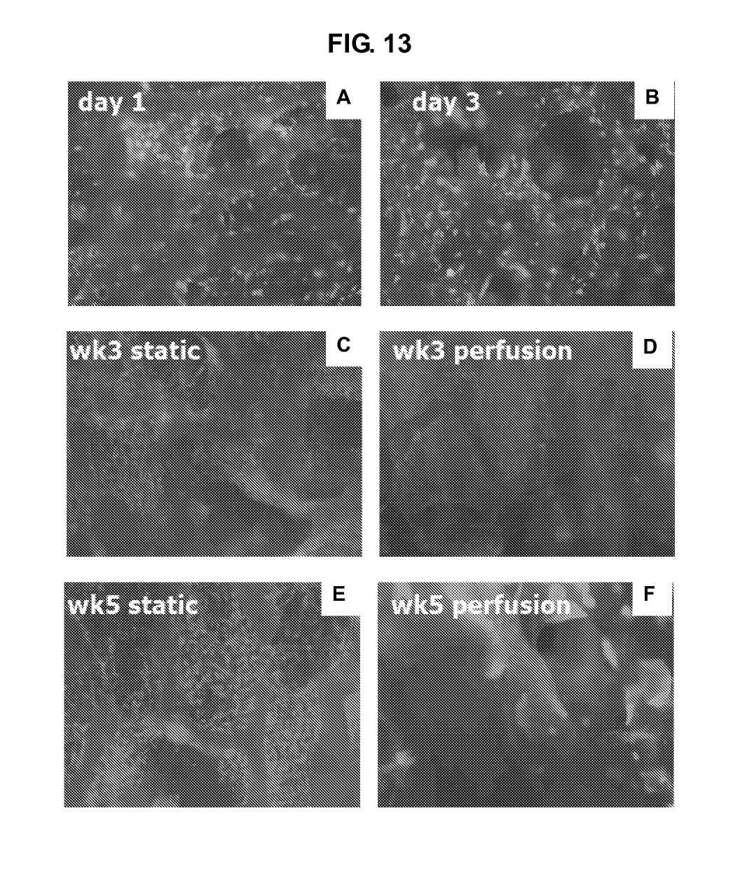

Fluorescent live-dead imaging shows cell survival of H9 progenitors in bone constructs (see e.g., FIG. 13A-F).

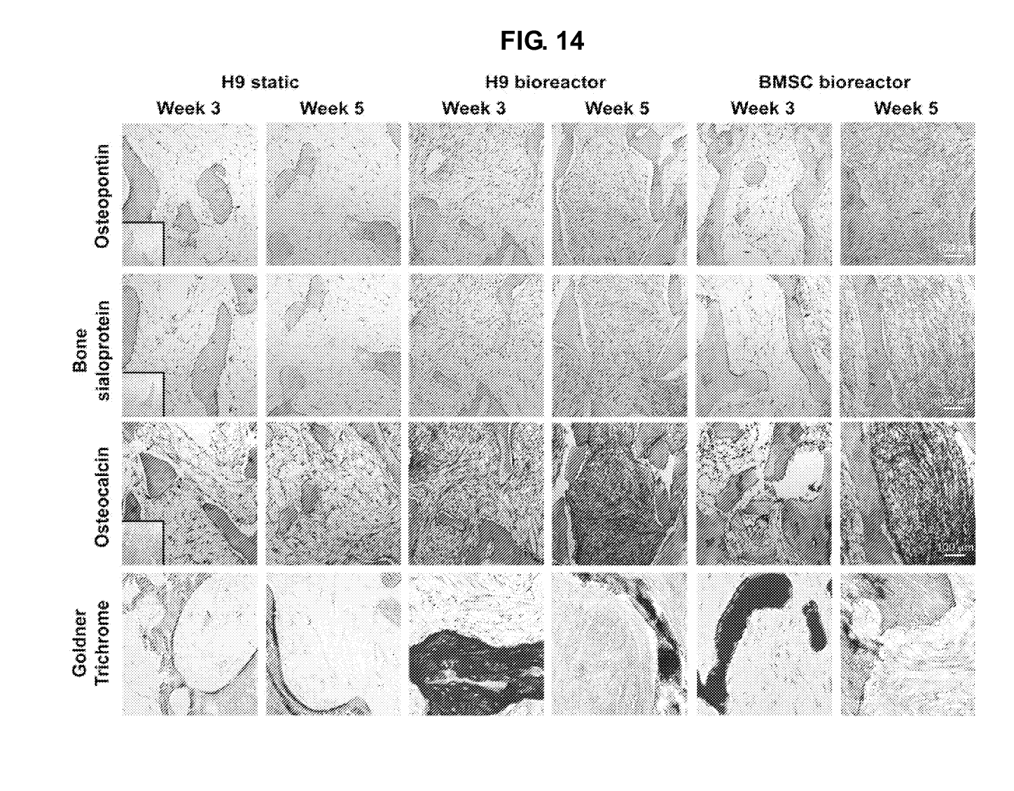

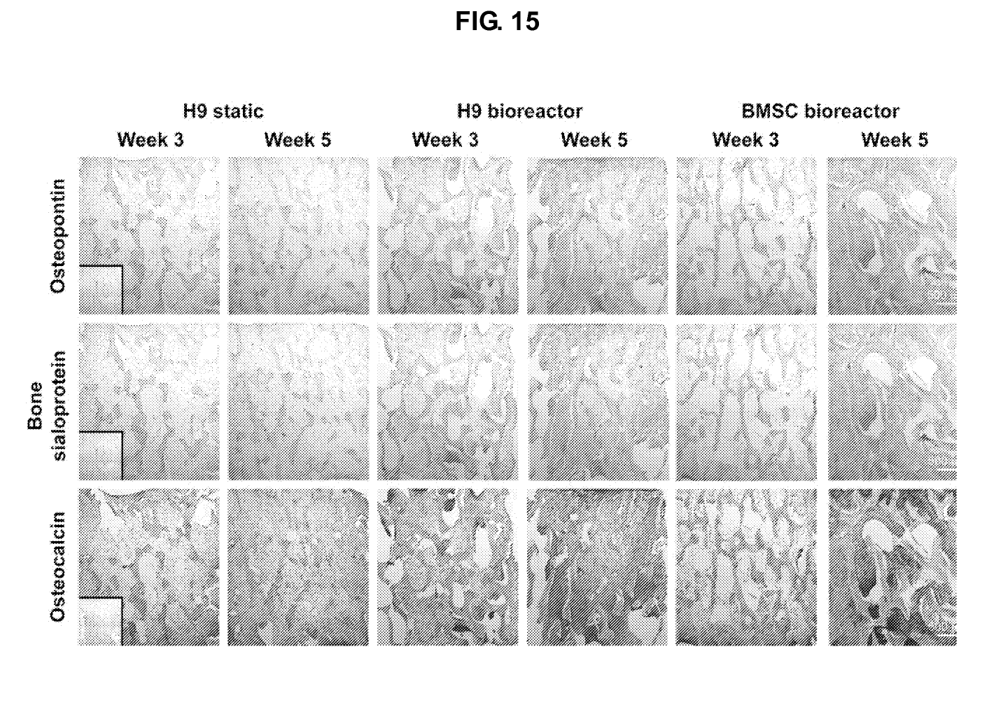

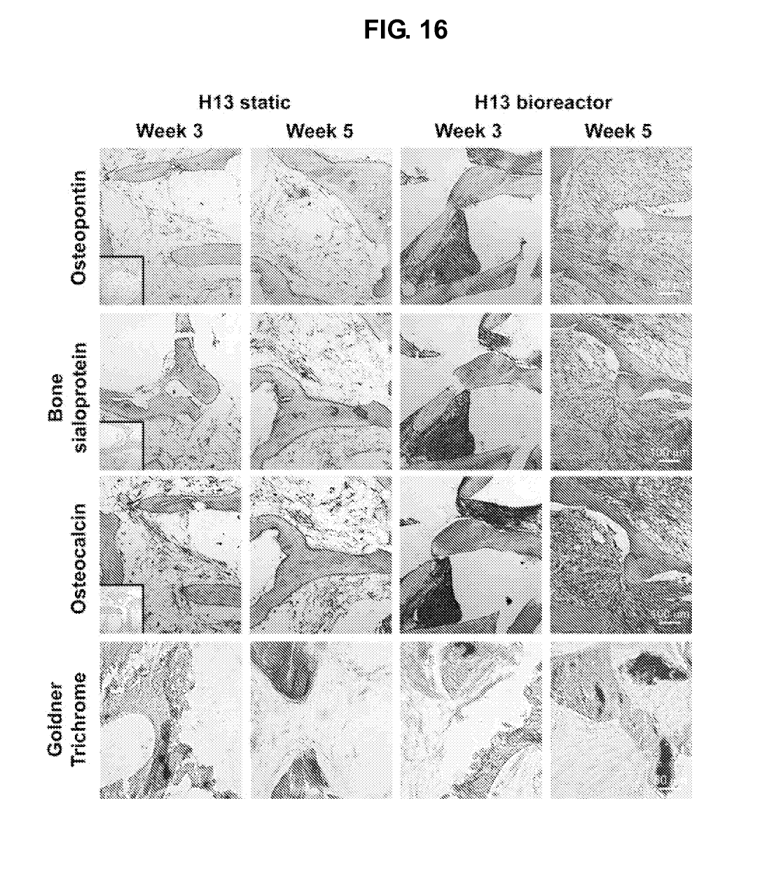

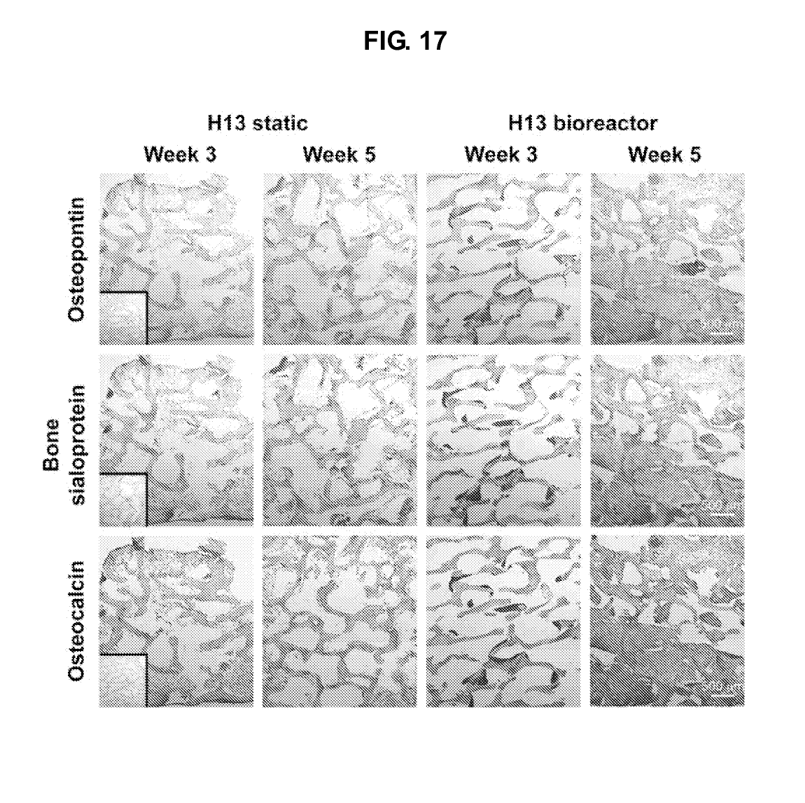

Histological examinations revealed the presence osteopontin, bone sialoprotein and osteocalcin in tissue engineered from hESC-progenitors and BMSC, indicating at the development and maturation of bone specific matrix (see e.g., FIG. 14 and FIG. 15). In static culture of H9 derived cells, only scarce bone protein matrix deposition was noted after 3 weeks and 5 weeks of culture, limited to the areas around the cells. In contrast, perfused cultures of H9-derived cells on bone scaffolds yielded much denser bone matrix at 3 weeks, which markedly increased in density to fill the pore spaces after 5 weeks of culture. Similar bone matrix deposition was noted in BMSC and hESC constructs in perfusion (see e.g., FIG. 14). Bone matrix was homogenously distributed through the scaffolding after 5 weeks of bioreactor culture (see e.g., FIG. 15). Reproducibility of bone matrix formation in perfusion bioreactors was corroborated in cultures of H13-progenitor constructs, which exhibited patchy but dense bone matrix after 3 weeks, and more homogenous dense matrix after 5 weeks, significantly higher compared to static culture (see e.g., FIG. 16 and FIG. 17).

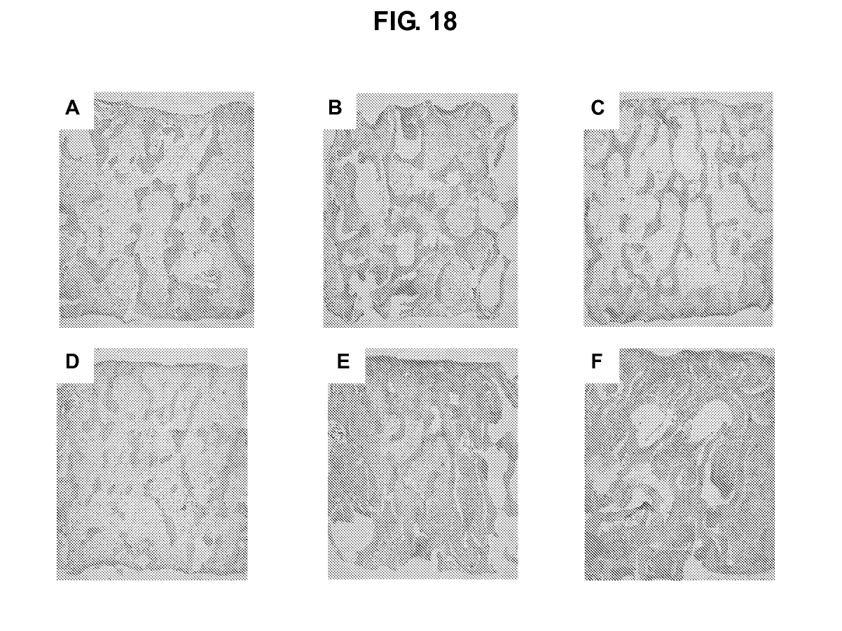

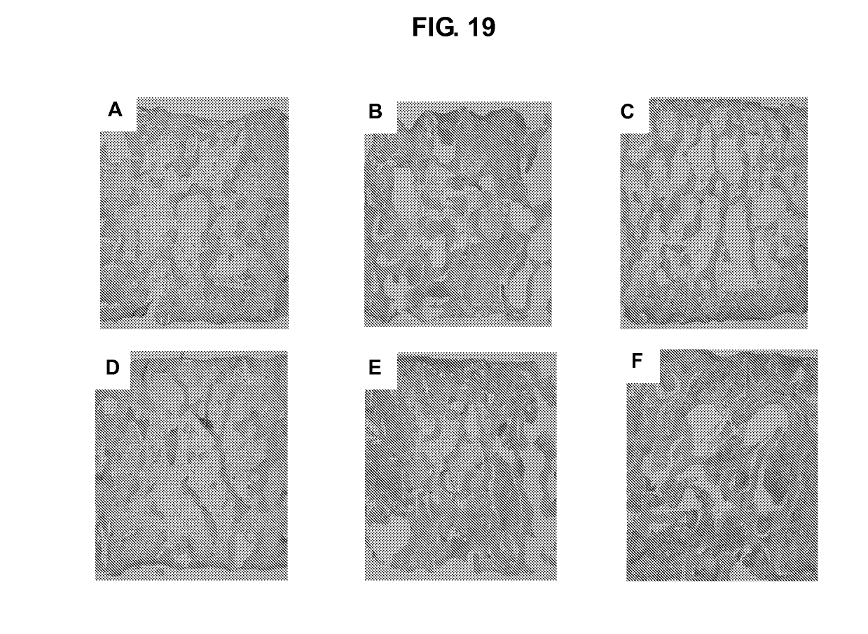

This example also shows bone tissue stability by homogeneous expression of bone markers in engineered tissue in H9 progenitors. Bioreactor-engineered tissue from H9-progenitors and BMSC stained strongly-positive for bone markers ostepontin (see e.g., FIG. 14 and FIG. 15, first row), bone sialoprotein (see e.g., FIG. 15, second row) and osteocalcin (see e.g., FIG. 14 and FIG. 15, third row), where insets represent negative staining controls. Minimal staining was observed in statically-cultured groups at weeks 3 and 5. After 3 weeks of culture, less homogenous matrix deposition was noted in BMSC compared to H9-progenitor bioreactor cultures. Histological examination revealed formation of dense extracellular matrix proteins osteopontin (see e.g., FIG. 18 and FIG. 14 and FIG. 15), bone sialoprotein and osteocalcin (see e.g., FIG. 19 and FIG. 14 and FIG. 15). Bioreactor-engineered tissue from H9-progenitors stained strongly positive for bone markers osteopontin (FIG. 14, first row), bone sialoprotein (FIG. 14 and FIG. 15, second row) and osteocalcin (FIG. 14 and FIG. 15, third row). Minimal staining was observed in statically-cultured groups at weeks 3 and 5. New osteoid deposition (FIG. 14, fourth row, red color) was noted in all groups, however the strongest deposition was noted after 3 weeks of culture in bioreactor groups (see e.g., FIG. 14).

Bioreactor-engineered tissue from H13 progenitors stained strongly positive for bone markers ostepontin (see e.g., FIG. 16, first row), bone sialoprotein (see e.g., FIG. 16, second row) and osteocalcin (see e.g., FIG. 16, third row). Insets represent negative staining controls. Minimal staining was observed in statically-cultured groups at weeks 3 and 5. New osteoid deposition (fourth row, red color) was noted in both groups after 3 weeks, and in the bioreactor group after 5 weeks of culture.

This example shows additional images at low-magnification of bioreactor-engineered tissue from H13 progenitors also showed strong positive staining for bone markers osteopontin (see e.g., FIG. 17, first row), bone sialoprotein (see e.g., FIG. 17, second row) and osteocalcin (see e.g., FIG. 17, third row). Insets represent negative staining controls. Minimal staining was observed in statically-cultured groups at weeks 3 and 5. Interestingly, after 3 weeks of culture, dense and less homogenous matrix deposition was noted in bioreactor cultures compared to 5 weeks of culture.

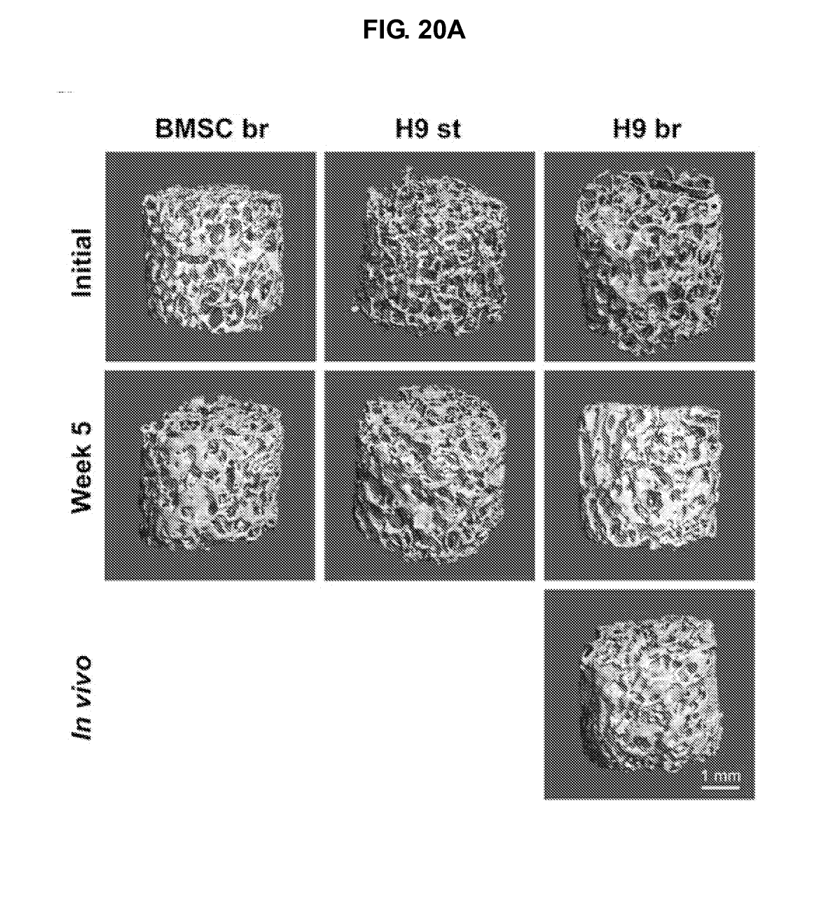

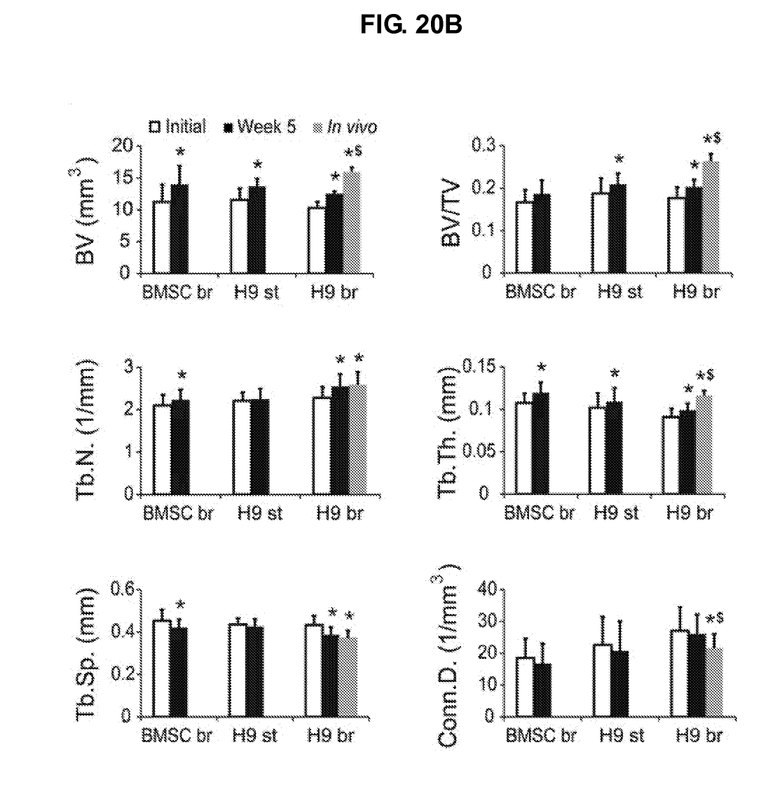

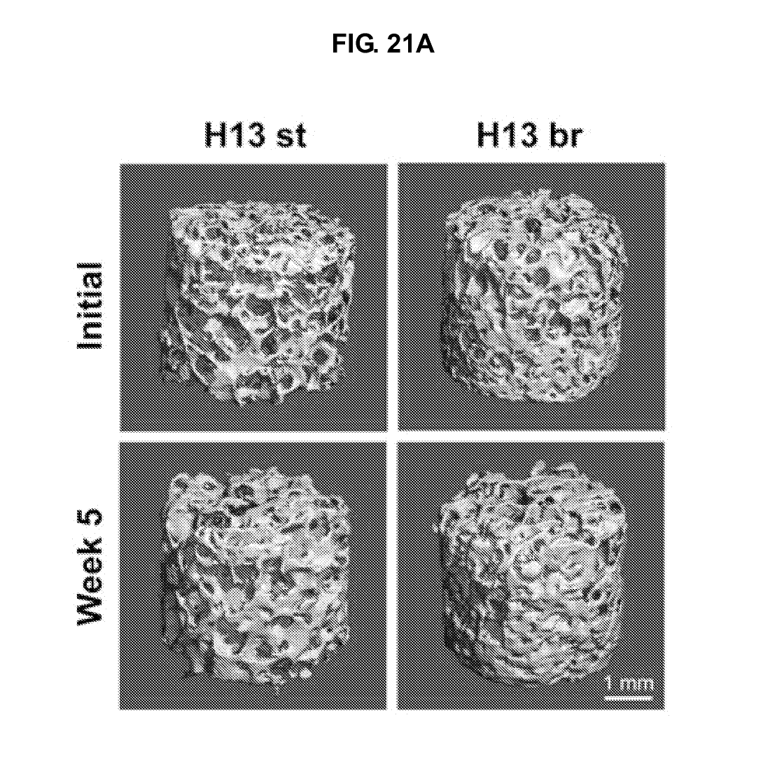

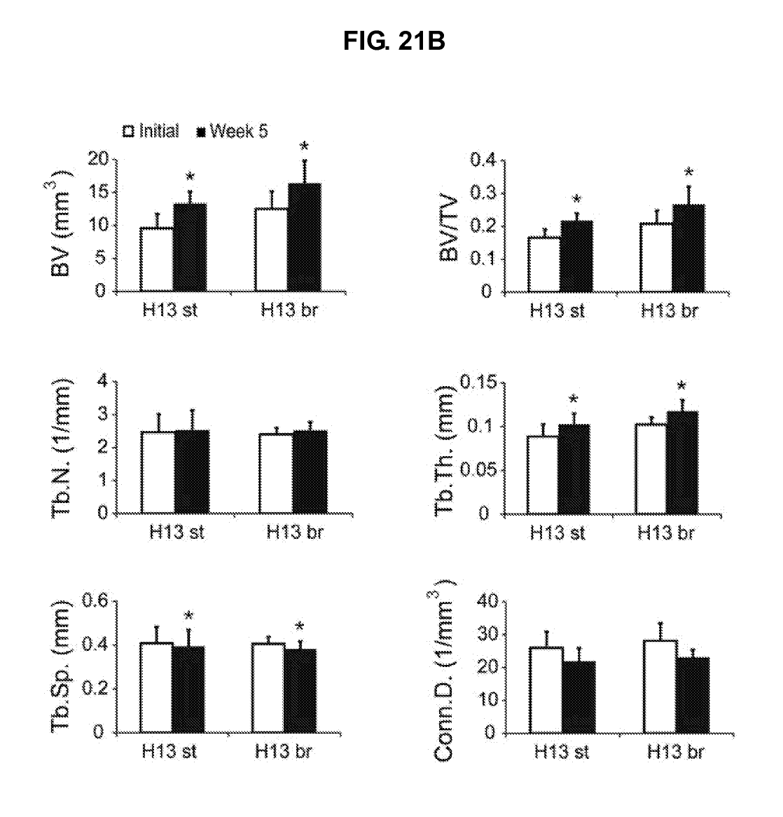

Formation of bone-like tissue was confirmed by positive Goldner's trichrome staining of osteoid (red color) in undecalcified engineered bone samples (see e.g., FIG. 14 and FIG. 16) and by μCT imaging of the mineralized tissue fraction. Significant increase in mineralized bone volume was observed, ratio of bone volume to total tissue volume, trabecular number and trabecular thickness in all three cell lines (see e.g., FIG. 20B). These changes were accompanied by significant decrease in trabecular spacing and by decreased connectivity density, indicating the increasing thickness and merging of the existing trabecular structures (see e.g., FIG. 20B). Similar results were obtained for bone engineered from H13-progenitors, showing the culture model supports formation of mature bone-like tissue (see e.g., FIG. 21).

Thus, the above example shows that compact bone grafts can be engineered from ESC-derived mesenchymal progenitors using the same scaffolds and bioreactor cultivation conditions as with BMSC and engineered bone properties were similar for the two cell sources. Also, importantly, stepwise ESC differentiation and bone engineering protocol yielded stable bone tissue with potential for further maturation and integration in vivo.

### Example 5

The following example shows engineered bone tissue remains stable in vivo with evidence of further maturation, vascularization and remodeling using an animal study of phenotypic stability and safety of engineered human bone (formed from H9 cells) by ectopic implantation (subcutaneous) of engineered constructs in immunocompromised mice.

Here, the progenitors were seeded into decellularized bone scaffolds at passage 4, and cultured for 5 weeks either statically or in a bioreactor with perfusion (interstitial flow) through the forming tissue. Bioreactor engineered-bone and control scaffolds seeded with hESC-progenitors or hESC were evaluated over 8 weeks of subcutaneous transplantation in SCID-beige mice for tissue stability and the absence of teratoma formation.

Initial macroscopic observations of tissue morphology indicate the absence of abnormal tissue growth in animals receiving engineered bone, in contrast to teratoma formation in animals receiving undifferentiated human embryonic stem cells delivered either in Matrigel (teratoma observed in 100% samples) or seeded in bone scaffolds (teratoma observed in 50% of the samples).

Cell viability was determined by a live/dead assay after seeding 3 and 5 weeks after culture. DNA content, alkaline phosphatase activity and osteopontin release into culture medium were measured to assess cell growth and osteogenesis. Bone tissue formation was assessed by H&E, Masson Trichrome, osteopontin, bone sialoprotein and osteocalcin staining, and by μCT imaging. Further evaluation of the properties and maturation of explanted tissues is shown below, using histological and immunohistochemical analyses and μCT.

Mesenchymal cells differentiated from hESC have been shown to form bone in vivo, however this was accompanied by formation of other tissue types and evidence of teratomas (Kuznetsov, 2011).

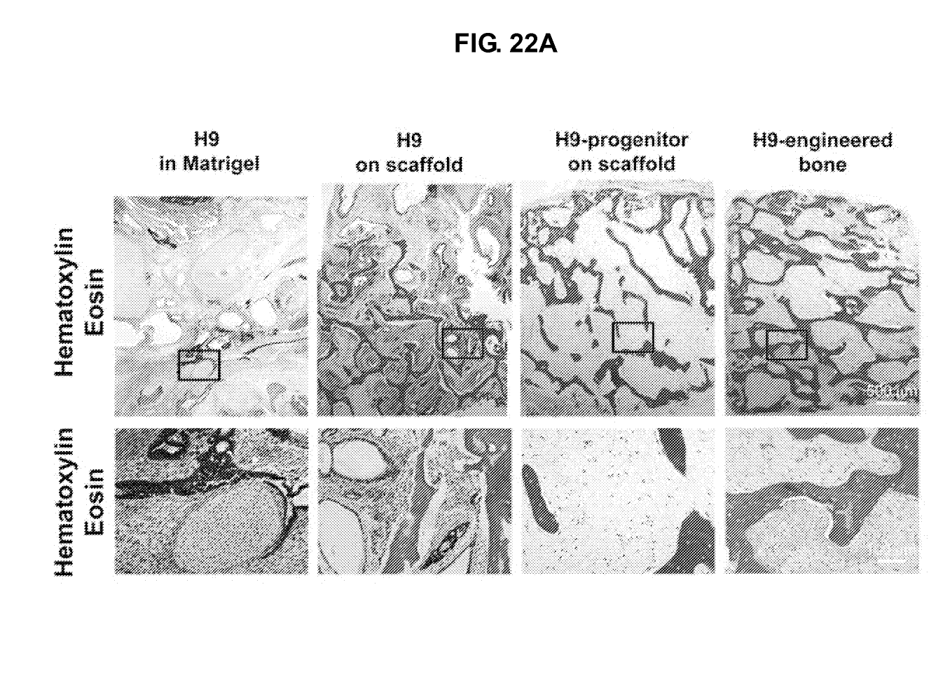

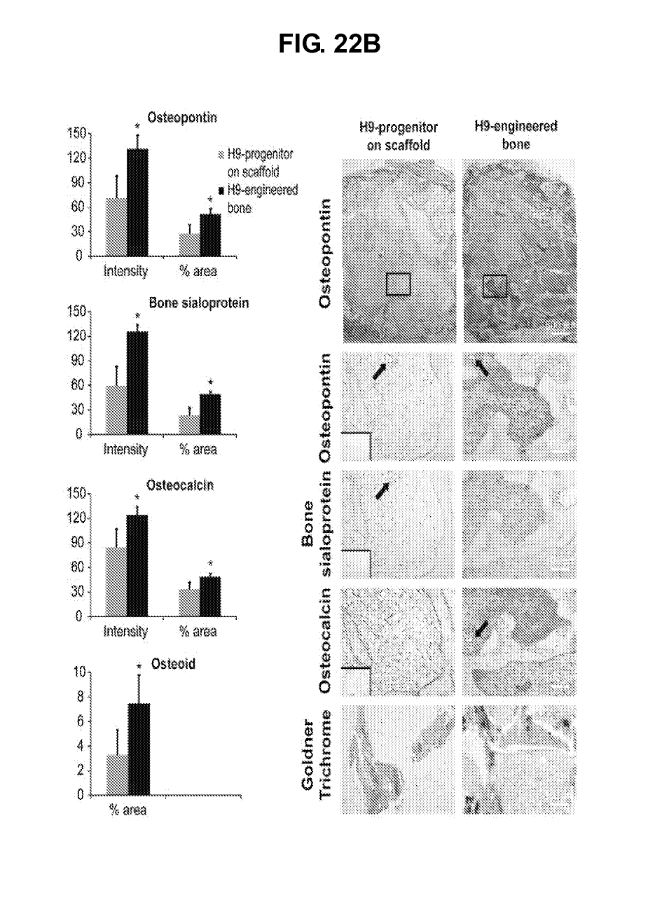

This example also shows the stability of engineered bone in vivo (see e.g., FIG. 22). Histological analysis indicated stability of mature bone phenotype in H9-engineered bone after 8 weeks of subcutaneous transplantation. After 8 weeks of subcutaneous implantation, further maturation of engineered bone was noted, resulting in denser bone matrix compared to scaffolds seeded with progenitors prior to implantation (see e.g., FIG. 22A). Here, undifferentiated cells invariably formed teratomas containing lineages of all three germ layers after 7 weeks in vivo (see e.g., FIG. 22A).

Importantly there was no evidence of teratoma tissue formation, which was found as expected in ESC-seeded scaffolds (see e.g., FIG. 22A). The scaffolds seeded with H9-progenitors did not form teratomas, but instead formed loose connective tissue that was weakly positive for bone matrix proteins (see e.g., FIG. 22A-B). In particular, there was no evidence of either endodermal or ectodermal layer tissues. In contrast, the engineered bone maintained bone matrix proteins (see e.g., FIG. 22B), with high density areas associated with scaffold structures. Engineered bone constructs contained microvasculature spanning interior regions of the scaffolds, and initiation of remodeling shown by the presence of osteoclastic cells in the outer regions. In contrast, formation of loose connective tissue was detected in bone scaffolds seeded with H9-progenitors prior to implantation, and teratoma tissue was found in bone scaffolds and Matrigel seeded with undifferentiated H9 cells (see e.g., FIG. 22A). Quantitative histomorphometric analyses of engineered bone indicated significantly higher staining intensity and % area stained positively for bone markers osteopontin, bone sialoprotein and osteocalcin compared to scaffolds seeded with H9-progenitors. Insets represent negative staining controls. Similarly, significantly higher % scaffold area covered with osteoid (red) was noted in engineered bone compared to scaffolds seeded with H9-progenitors (see e.g., FIG. 22B). This example indicates that the bioreactor cultivation step enhances the stability and maturation of engineered bone tissue.

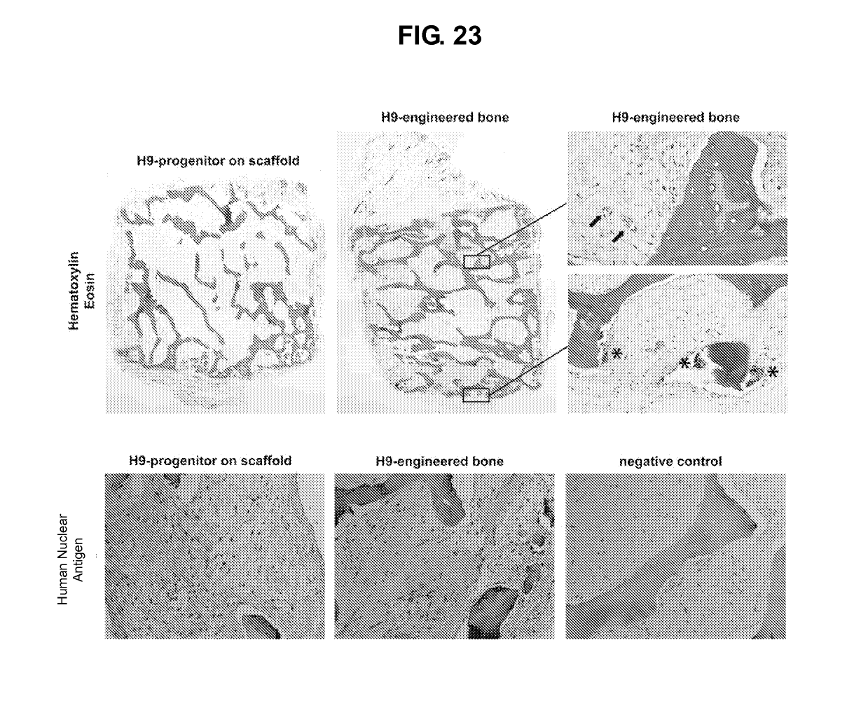

This example shows further examination of engineered bone tissue after explantation. Additionally, cell lineages not normally preset in bone tissue were not detected within the scaffold structure (see e.g., FIG. 23). The explants were surrounded by loose connective tissue capsules and contained functional microvasculature evidenced by the presence of red blood cells in the interior of engineered tissue. Interestingly, osteoclast invasion at the construct edges initiating the remodeling process of the scaffold was also observed (see e.g., FIG. 23). Scaffolds seeded with H9 progenitors and H9-engineered bone exhibited loose connective tissue and denser bone-like tissue upon closer examination. At higher magnification (see e.g., FIG. 23, top right), the presence of functional microvessels containing red blood cells was evident in the edge and interior regions of the scaffold (marked by arrows). In addition, external regions of the scaffolds indicated the initiation of remodeling process, evidenced by invasion of multinuclear osteoclastic cells overlaying and degrading scaffold surfaces (marked by asterisks). Human origin of the cells was confirmed by positive staining of human nuclear antigen (see e.g., FIG. 23, bottom) in both scaffolds seeded with H9-progenitors as well as H9-engineered bone (brown color).

This example shows bone mineralization measurements with μCT in H13 progenitors in static and bioreactor conditions. Reconstructed 3D μCT images allowed for the measurement of bone volume, bone volume fraction and trabecular thickness. Each of these parameters increased significantly in both groups, in contrast to trabecular spacing which decreased significantly in both groups, indicating bone maturation. μCT revealed bone mineralization tissue formation during the 5-week culture in all groups (see e.g., FIG. 20). Osteogenesis and bone tissue formation were comparable for ESC and BMSC. Bone volume (BV), bone volume fraction (BV/TV), trabecular number (Tb.N.) and trabecular thickness (Tb.Th.) increased significantly in the H9 bioreactor group, in contrast to trabecular spacing (Tb.Sp.) and connectivity density (Conn.D.) which exhibited a decreasing trend. Similar changes in mineralized tissue were noted for the H9 static and BMSC bioreactor groups (see e.g., FIG. 20B).

The μCT examination of the constructs explanted after 8 weeks revealed continued maturation of the mineralized bone matrix, with increasing maturation of the mineralized matrix, including significantly higher bone volume, significantly higher bone volume to tissue volume ratio and significantly higher trabecular thickness compared to engineered bone after 5 weeks of culture (see e.g., FIG. 20B).

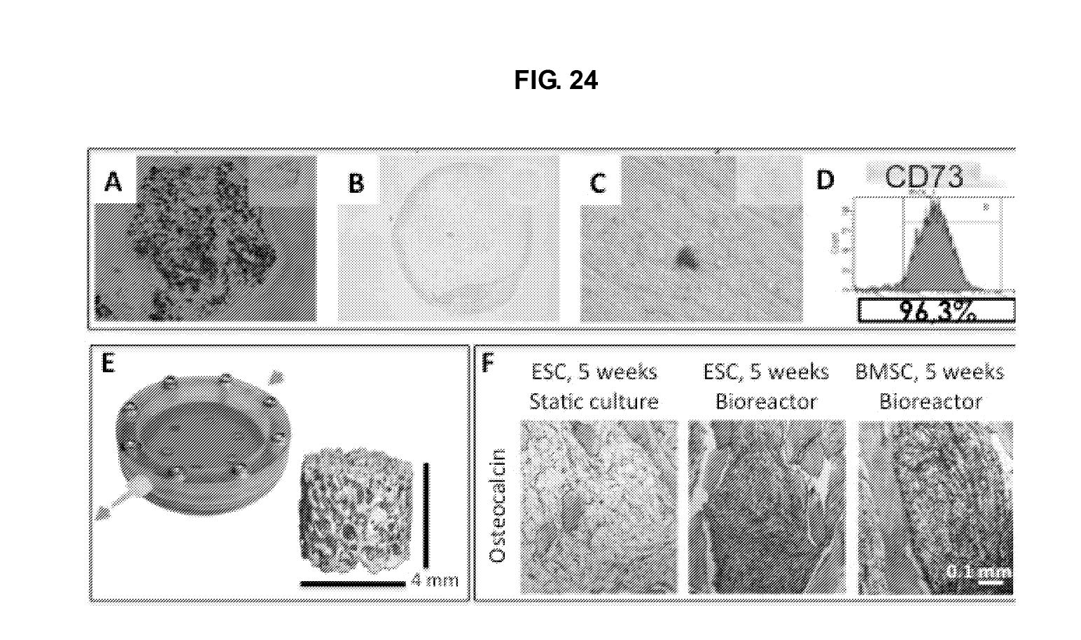

In conclusion, this example shows the successfully applied perfusion model to engineer bone from human embryonic stem cells (see e.g., FIG. 24). This example shows that culture parameters supporting bone formation from BMSC and ASC can be translated to mesenchymal-like progenitors derived from pluripotent stem cells. This example shows differentiation of two lines of ESC(H9, H13, Wicell Research Institute) and characterized mesenchymal-like properties of the obtained progenitors (expression of surface antigens; potentials for osteogenesis, chondrogenesis, adipogenesis) (see e.g., FIG. 24A-D). In this example, ESC-progenitors were seeded on decellularized bovine bone scaffolds and cultured in the constructs using perfusion conditions for 5 weeks (see e.g., FIG. 24E). Both ESC lines show significantly higher cell numbers and denser bone-like tissue in bioreactors compared to statically-cultured controls (see e.g., FIG. 24F). Engineered bone remained stable upon subsequent implantation into the backs of immunocompromised mice (ectopic site) for 8 weeks.

Thus, compared to the bone scaffolds seeded with progenitors before implantation, significantly denser bone extracellular matrix was observed in the engineered bone group.

### REFERENCES

Kuznetsov S A, Cheman N, Robey P G. Stem Cells Dev. 2010 Sep. 14. [Epub ahead of print]. In Vivo Bone Formation by Progeny of Human Embryonic Stem Cells.
Hu J, Smith L A, Feng K, Liu X, Sun H, Ma P X. Tissue Eng Part A. 2010 Jul. 2. [Epub ahead of print] Response of Human Embryonic Stem Cells Derived Mesenchymal Stem Cells to Osteogenic Factors and Architectures of Materials during in vitro Osteogenesis.
Smith L A, Liu X, Hu J, Ma P X. The enhancement of human embryonic stem cell osteogenic differentiation with nano-fibrous scaffolding. Biomaterials. 2010 July; 31(21):5526-35. Epub 2010 Apr. 28.
Harkness L, Mahmood A, Ditzel N, Abdallah B M, Nygaard J V, Kassem M. Bone. 2010 Sep. 30. [Epub ahead of print] Selective isolation and differentiation of a stromal population of human embryonic stem cells with osteogenic potential.
Tian X F, Heng B C, Ge Z, Lu K, Rufaihah A J, Fan V T, Yeo J F, Cao T. Comparison of osteogenesis of human embryonic stem cells within 2D and 3D culture systems. Scand J Clin Lab Invest. 2008; 68(1):58-67.
Mateizel I, De Becker A, Van de Velde H, De Rycke M, Van Steirteghem A, Cornelissen R, Van der Elst J, Liebaers I, Van Riet I, Sermon K. Efficient differentiation of human embryonic stem cells into a homogeneous population of osteoprogenitor-like cells. Reprod Biomed Online. 2008 May; 16(5):741-53.
Boyd N L, Robbins K R, Dhara S K, West F D, Stice S L. Human embryonic stem cell-derived mesoderm-like epithelium transitions to mesenchymal progenitor cells. Tissue Eng Part A. 2009 August; 15(8):1897-907.
Hwang N S, Varghese S, Elisseeff J. Derivation of chondrogenically-committed cells from human embryonic cells for cartilage tissue regeneration. PLoS One. 2008 Jun. 25; 3(6):e2498.
Hwang N S, Varghese S, Lee H J, Zhang Z, Ye Z, Bae J, Cheng L, Elisseeff J. In vivo commitment and functional tissue regeneration using human embryonic stem cell-derived mesenchymal cells. Proc Natl Acad Sci USA. 2008 Dec. 30; 105(52):20641-6. Epub 2008 Dec. 18.

What is claimed is:

1. A method of forming a bone tissue module comprising:
inducing differentiation of progenitor cells to form osteogenic progenitor cells;
expanding the osteogenic progenitor cells;
combining the osteogenic progenitor cells and a biocompatible scaffold comprising a matrix material; and
incubating the osteogenic progenitor cells and the biocompatible scaffold so as to form a bone tissue module.

2. The method of claim 1, wherein:
inducing differentiation of osteogenic progenitor cells to form osteogenic progenitor cells comprises inducing differentiation of embryonic stem cells (ESCs) to form mesenchymal-like progenitor cells;
expanding the osteogenic progenitor cells comprises expanding the mesenchymal-like progenitor cells;
combining the osteogenic progenitor cells and a biocompatible scaffold comprising a matrix material comprises combining the expanded mesenchymal-like progenitor cells and a biocompatible scaffold comprising a matrix material; and
incubating the osteogenic progenitor cells and the biocompatible scaffold so as to form a bone tissue module comprises incubating the expanded mesenchymal-like progenitor cells and the biocompatible scaffold so as to form a bone tissue module.

3. The method of any one of claims 1-2, wherein incubating comprises incubating the osteogenic progenitor cells and the biocompatible scaffold in vitro in a bioreactor.

4. The method of any one of claims 1-3, wherein the progenitor cells or the osteogenic progenitor cells comprise cells selected from the group consisting of mesenchymal stem cells (MSC), MSC-derived cells, embryonic stem cells, bone marrow stromal/stem cells, osteoblasts, and induced pluripotent cell lines.

5. The method of any one of claims 1-4, wherein the progenitor cells comprise embryonic stem cells or induced pluripotent stem cells.

6. The method of any one of claims 1-5, wherein the progenitor cells comprise human progenitor cells.

7. The method of any one of claims 1-6, wherein the matrix comprises decellularized bone.

8. The method of any one of claims 1-7, wherein the matrix comprises a material selected from the group consisting of fibrin, fibrinogen, a collagen, a polyorthoester, a polyvinyl alcohol, a polyamide, a polycarbonate, a polyvinyl pyrrolidone, a marine adhesive protein, a cyanoacrylate, a polymeric hydrogel, and a combination thereof.

9. The method of any one of claims 1-8, wherein the biocompatible scaffold comprises progenitor cells at a density of at least about 0.0001 million cells (M) ml⁻¹ up to about 1000 M ml⁻¹.

10. The method of any one of claims 1-9, wherein the biocompatible scaffold comprises progenitor cells at a density of about 1 M ml⁻¹, about 5 M ml⁻¹, about 10 M ml⁻¹, about 15 M ml⁻¹, about 20 M ml⁻¹, about 25 M ml⁻¹, about 30 M ml⁻¹, about 35 M ml⁻¹, about 40 M ml⁻¹, about 45 M ml⁻¹, about 50 M ml⁻¹, about 55 M ml⁻¹, about 60 M ml⁻¹, about 65 M ml⁻¹, about 70 M ml⁻¹, about 75 M ml⁻¹, about 80 M ml⁻¹, about 85 M ml⁻¹, about 90 M ml⁻¹, about 95 M ml⁻¹, or about 100 M ml⁻¹.

11. The method of any one of claims 1-10, wherein the biocompatible scaffold comprises progenitor cells at a density of at least about 30 M ml⁻¹.

12. The method of any one of claims 1-11, wherein inducing differentiation of progenitor cells comprises incubating progenitor cells in a differentiation medium.

13. The method of claim 12, wherein the differentiation medium comprises DMEM, serum, dexamethasone, b-glycerophosphate, ascorbic acid, bone morphogenic protein, vitamin D, and pen-strep.

14. The method of any one of claims 1-13, wherein expanding the osteogenic progenitor cells comprises culturing the osteogenic progenitor cells in an expansion medium.

15. The method of claim 14, wherein the expansion medium comprises DMEM, serum or KO-serum replacement, nonessential amino acids, glutamine, b-mercaptoethanol, and bFGF.

16. The method of any one of claims 1-15, wherein incubating the osteogenic progenitor cells and the biocompatible scaffold comprises incubating the osteogenic progenitor cells and the biocompatible scaffold in vitro in a bioreactor at a superficial flow velocity of (i) between about 80 μm/s and about 2,000 μm/s or (ii) about 80 μm/s, about 100 μm/s, about 200 μm/s, about 300 μm/s, about 400 μm/s, about 500 μm/s, about 600 μm/s, about 700 μm/s, about 800 μm/s, about 900 μm/s, about 1,000 μm/s, about 1,100 μm/s, about 1,200 μm/s, about 1,300 μm/s, about 1,400 μm/s, about 1,500 μm/s, about 1,600 μm/s, about 1,700 μm/s, about 1,800 μm/s, about 1,900 μm/s, or about 2,000 μm/s.

17. The method of any one of claims 1-16, wherein incubating the osteogenic progenitor cells and the biocompatible scaffold comprises incubating the osteogenic progenitor cells and the biocompatible scaffold in vitro in a bioreactor at a superficial flow velocity of about 400 μm/s to about 800 μm/s.

18. The method of any one of claims 1-17, wherein incubating the osteogenic progenitor cells and the biocompatible scaffold comprises incubating the osteogenic progenitor cells and the biocompatible scaffold in an osteogenic medium.

19. The method of claim 18, wherein the osteogenic medium comprises DMEM, FBS, beta-glycerophosphate, dexamethasone and ascorbate-2 phosphate.

20. The method of claim 18, wherein the osteogenic medium comprises DMEM, dexamethasone, ascorbate-2 phosphate, proline, ITS supplement, sodium pyruvate, TGFB3, and pen-strep.

21. A method of treating a bone tissue defect comprising grafting a bone tissue module produced according to any one of claims 1-20 into a subject in need thereof.

22. The method of claim 21, wherein the subject is a mammalian subject.

23. The method of claim 21 wherein the subject is a horse, cow, dog, cat, sheep, pig, rabbit, goat, chicken, or human.

24. The method of any one of claims 21-23, wherein the bone tissue defect comprises at least one of arthritis; osteoarthritis; osteoporosis; osteochondrosis; osteochondritis; osteogenesis imperfecta; osteomyelitis; osteophytes; achondroplasia; costochondritis; chondroma; chondrosarcoma; herniated disk; Klippel-Feil syndrome; osteitis deformans; osteitis fibrosa cystica, a congenital defect that results in absence of a tissue; accidental tissue defect or damage; fracture; wound; joint trauma; an autoimmune disorder; diabetes; Charcot foot; cancer; tissue resection; periodontal disease; implant extraction; or tumor resection.

25. The method of any one of claims 21-24, wherein the bone tissue module does not induce any substantially abnormal growth in the subject.
